(19) United States
(12) Patent Application Publication
(10) Pub. No.: US 2009/0093473 A1
(43) Pub. Date: Apr. 9, 2009

(54) Spiro-benzimidazoles as inhibitors of gastric acid secretion

(76) Inventors: Peter Jan Zimmermann, Ehningen (DE); Jorg Senn-Bilfinger, Konstanz (DE); Christof Brehm, Mannheim (DE); Wilm Buhr, Konstanz (DE); Maria Vittoria Chiesa, Konstanz (DE); Andreas Palmer, Singen (DE); Wolfgang-Alexander Simon, Konstanz (DE); Stefan Postius, Konstanz (DE); Wolfgang Kromer, Konstanz (DE)

Correspondence Address: NATH & ASSOCIATES PLLC, 112 South West Street, Alexandria, VA, 22314, US

(21) Appl. No.: 11/921,508

(22) PCT Filed: Jun. 13, 2006
(86) PCT No.: PCT/EP2006/063163

§ 371 Date: Dec. 4, 2007

(30) Foreign Application Priority Data
Jun. 16, 2005 (EP) 05105330.4

(51) Int. Cl. A61K 31/438; C07D 471/10; A61P 1/00; A61K 31/5377

(52) U.S. Cl. 514/232.8; 546/18; 544/70; 514/278

(57) ABSTRACT

The invention provides compounds of the formula (I)

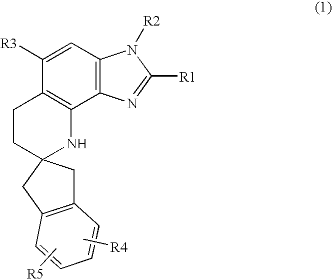

In which the substituents and symbols are as defined in the description. The compounds inhibit the secretion of gastric acid.

### TECHNICAL FIELD

The invention relates to novel compounds which are used in the pharmaceutical industry as active compounds for the production of medicaments.

### BACKGROUND ART

In European patent application 266326 (which corresponds to U.S. Pat. No. 5,106,862), benzimidazole derivatives having a very broad variety of substituents are disclosed, which are said to be active as anti-ulcer agents. In the International Patent Application WO 97/47603 (Astra AB) benzimidazoles with a specific benzyloxy or benzylamino substitution are described.

The International Patent Application WO 04/054984 discloses substituted, bicyclic benzimidazole derivatives which compounds are useful for treating gastrointestinal diseases.

The International Patent Application WO 04/087701 discloses tricyclic benzimidazole derivatives having different substituents in 5-position of the benzimidazole moiety which compounds are likewise useful for treating gastrointestinal diseases.

The International Patent Applications WO 05/058893 and WO 05/103057 disclose tricyclic benzimidazole derivatives having substituents in 6- and 7-position of the tricyclic ring system which compounds are likewise useful for treating gastrointestinal diseases.

The International Patent Application WO 05/121139 discloses tricyclic benzimidazole derivatives having substituents in 5-, 6- and 7-position of the tricyclic ring system which compounds are likewise useful for treating gastrointestinal diseases.

### DISCLOSURE OF INVENTION

### Technical Problem

A whole series of compounds are known from the prior art which inhibit gastric acid secretion by blockade of the H+/K+-ATPase. The compounds designated as proton pump inhibitors (PPI's), for example omeprazole, esomeprazole, lansoprazole, pantoprazole or rabeprazole, bind irreversibly to the H+/K+-ATPase. PPI's are available as therapeutics for a long time already. A new class of compounds designated as reversible proton pump inhibitors (rPPI's), as acid pump antagonists (APA's) or as potassium competitive acid blockers (P-CAB's) bind reversibly to the H+/K+-ATPase. Although rPPI's, APA's and P-CAB's are known for more than 20 years and many companies are engaged in their development, no rPPI, APA or P-CAB is at present available for therapy. The technical problem underlying the present invention is therefore to provide acid pump antagonists which can be used in therapy.

### Technical Solution

The invention relates to compounds of the formula 1

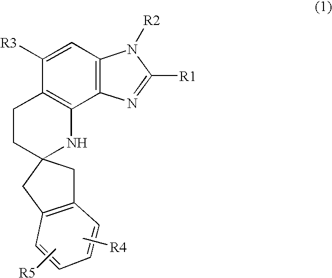

in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 1-4C-alkoxy, 3-7C-cycloalkyl, 3-7C-cycloalkyl-1-4C-alkyl, hydroxy-1-4C-alkyl, 1-4C-alkoxy-1-4C-alkyl, 2-4C-alkenyl, 2-4C-alkynyl or fluoro-1-4C-alkyl,
R3 is hydrogen, halogen, fluoro-1-4C-alkyl, 1-4C-alkyl, 2-4C-alkenyl, 2-4C-alkynyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32, where
   R31 is hydrogen, hydroxyl, 1-7C-alkyl, 3-7C-cycloalkyl, hydroxy-1-4C-alkyl, 1-4C-alkoxy-1-4C-alkyl, 1-4C-alkylcarbonyl-1-4C-alkyl, 1-4C-alkylcarbonyl or 1-4C-alkoxycarbonyl and
   R32 is hydrogen, 1-7C-alkyl, hydroxy-1-4C-alkyl, or 1-4C-alkoxy-1-4C-alkyl, or where
   R31 and R32 together, including the nitrogen atom to which both are bonded, are a pyrrolidino, hydroxypyrrolidino, piperidino, piperazino, azetidino, hydroxyazetidino, fluorazetidino, aziridino, N-1-4C-alkylpiperazino or morpholino group,
R4 and R5 are identical or different substituents selected from the group consisting of hydrogen, 1-4C-alkyl, hydroxy-1-4C-alkyl, 1-4C-alkoxy, 1-4C-alkoxy-1-4C-alkyl, hydroxy-1-4C-alkoxy, 1-4C-alkoxy-1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, trifluoromethyl, halo-1-4C-alkoxy, nitro, amino, mono- or di-1-4C-alkylamino, 1-4C-alkyl-carbonylamino, 1-4C-alkoxycarbonylamino, 1-4C-alkoxy-1-4C-alkoxycarbonylamino or sulfonyl,
   and their salts.

1-4C-Alkyl represents straight-chain or branched alkyl groups having 1 to 4 carbon atoms. Examples which may be mentioned are the butyl, isobutyl, sec-butyl, tert-butyl, propyl, isopropyl, ethyl and the methyl group.

3-7C-Cycloalkyl represents cyclopropyl, cyclobutyl, cyclopentyl, cyclohexyl and cycloheptyl, of which cyclopropyl, cyclobutyl and cyclopentyl are preferred.

3-7C-Cycloalkyl-1-4C-alkyl represents one of the aforementioned 1-4C-alkyl groups, which is substituted by one of the aforementioned 3-7C-cycloalkyl groups. Examples which may be mentioned are the cyclopropylmethyl, the cyclohexylmethyl and the cyclohexylethyl group.

1-4C-Alkoxy represents groups, which in addition to the oxygen atom contain a straight-chain or branched alkyl group having 1 to 4 carbon atoms. Examples which may be mentioned are the butoxy, isobutoxy, sec-butoxy, tert-butoxy, propoxy, isopropoxy and preferably the ethoxy and methoxy group.

1-4C-Alkoxy-1-4C-alkyl represents one of the aforementioned 1-4C-alkyl groups, which is sub-stituted by one of the aforementioned 1-4C-alkoxy groups. Examples which may be mentioned are the methoxymethyl group, the methoxyethyl group, in particular the 2-methoxyethyl group, the ethoxyethyl group, in particular the 2-ethoxyethyl group, and the butoxyethyl group, in particular the 2-butoxyethyl group.

1-4C-Alkoxycarbonyl (—CO-1-4C-alkoxy) represents a carbonyl group, to which one of the aforementioned 1-4C-alkoxy groups is bonded. Examples which may be mentioned are the methoxycarbonyl (CH₃O—C(O)—) and the ethoxycarbonyl group (CH₃CH₂O—C(O)—).

2-4C-Alkenyl represents straight-chain or branched alkenyl groups having 2 to 4 carbon atoms. Examples which may be mentioned are the 2-butenyl, 3-butenyl, 1-propenyl and the 2-propenyl group (allyl group).

2-4C-Alkynyl represents straight-chain or branched alkynyl groups having 2 to 4 carbon atoms. Examples which may be mentioned are the 2-butynyl, 3-butynyl, and preferably the 2-propynyl, group (propargyl group).

Fluoro-1-4C-alkyl represents one of the aforementioned 1-4C-alkyl groups, which is substituted by one or more fluorine atoms. Examples which may be mentioned are the trifluoromethyl group, the difluoromethyl, the 2-fluoroethyl, the 2,2-difluoroethyl or the 2,2,2-trifluoroethyl group.

Hydroxy-1-4C-alkyl represents one of the aforementioned 1-4C-alkyl groups, which is substituted by a hydroxy group. Examples which may be mentioned are the hydroxymethyl, the 2-hydroxyethyl, the 3-hydroxypropyl, the (2S)-2-hydroxypropyl and the (2R)-2-hydroxypropyl group. Hydroxy-1-4C-alkyl within the scope of the invention is understood to include 1-4C-alkyl groups substituted by two or more hydroxy groups. Examples which may be mentioned are the 3,4-dihydroxybutyl and in particular the 2,3-dihydroxypropyl groups.

Halogen within the meaning of the invention is bromo, chloro and fluoro.

1-4C-Alkoxy-1-4C-alkoxy represents one of the aforementioned 1-4C-alkoxy groups, which is substituted by a further 1-4C-alkoxy group. Examples which may be mentioned are the groups 2-(methoxy)ethoxy (CH₃—O—CH₂—CH₂—O—) and 2-(ethoxy)ethoxy (CH₃—CH₂—O—CH₂—CH₂—O—).

1-4C-Alkoxy-1-4C-alkoxy-1-4C-alkyl represents one of the aforementioned 1-4C-alkoxy-1-4C-alkyl groups, which is substituted by one of the aforementioned 1-4C-alkoxy groups. An example which may be mentioned is the group 2-(methoxy)ethoxymethyl (CH₃—O—CH₂—CH₂—O—CH₂—).

Fluoro-1-4C-alkoxy-1-4C-alkyl represents one of the aforementioned 1-4C-alkyl groups, which is substituted by a fluoro-1-4C-alkoxy group. Fluoro-1-4C-alkoxy in this case represents one of the aforementioned 1-4C-alkoxy groups, which substituted by one or more fluorine atoms. Examples of fluoro-substituted 1-4C-alkoxy groups which may be mentioned are the 2-fluoroethoxy, 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-trifluoroethoxy, in particular the 1,1,2,2-tetrafluoroethoxy, the 2,2,2-trifluoroethoxy, the trifluoromethoxy and preferably the difluoromethoxy group. Examples of fluoro-1-4C-alkoxy-1-4C-alkyl radicals which may be mentioned are, 1,1,2,2-tetrafluoroethoxymethyl, the 2,2,2-trifluoroethoxymethyl, the trifluoromethoxymethyl, 2-fluoroethoxyethyl, the 1,1,2,2-tetrafluoroethoxyethyl, the 2,2,2-trifluoroethoxyethyl, the trifluoromethoxyethyl and preferably the difluoromethoxymethyl and the difluoromethoxyethyl radicals.

1-4C-Alkylcarbonyl represents a group, which in addition to the carbonyl group contains one of the aforementioned 1-4C-alkyl groups. An example which may be mentioned is the acetyl group.

1-4C-Alkylcarbonyl-1-4C-alkyl represents aforementioned 1-4C-alkyl groups which are substituted by 1-4C-alkylcarbonyl group. Examples which may be mentioned are the 2-oxo-propyl, the 2-oxo-butyl, the 2-oxo-pentyl, the 3-oxo-butyl or the 3-oxo-pentyl radicals.

Hydroxy-1-4C-alkoxy represents aforementioned 1-4C-alkoxy groups, which are substituted by a hydroxy group. A preferred example which may be mentioned is the 2-hydroxyethoxy group.

2-4C-Alkenyloxy represents groups, which in addition to the oxygen atom contain one of the abovementioned 2-4C-alkenyl groups. Examples, which may be mentioned, are the 2-butenyloxy, 3-butenyloxy, 1-propenyloxy and the 2-propenyloxy group (allyloxy group).

Carboxy-1-4C-alkyl represents 1-4C-alkyl groups which are substituted by a carboxyl group. Examples, which may be mentioned, are the carboxymethyl and the 2-carboxyethyl group.

1-4C-Alkoxycarbonyl-1-4C-alkyl represents 1-4C-alkyl groups, which are substituted by one of the abovementioned 1-4C-alkoxycarbonyl groups. Examples, which may be mentioned, are the Methoxycarbonylmethyl and the ethoxycarbonylmethyl group.

Halo-1-4C-alkoxy represents 1-4C-alkoxy groups which are completely or mainly substituted by halogen. “Mainly” in this connection means that more than half of the hydrogen atoms in the 1-4C-alkoxy groups are replaced by halogen atoms. Halo-1-4C-alkoxy groups are primarily chloro- and/or in particular fluoro-substituted 1-4C-alkoxy groups. Examples of halogen-substituted 1-4C-alkoxy groups which may be mentioned are the 2,2,2-trichloroethoxy, the hexachloroisopropoxy, the pentachloroisopropoxy, the 1,1,1-trichloro-3,3,3-trifluoro-2-propoxy, the 1,1,1-trichloro-2-methyl-2-propoxy, the 1,1,1-trichloro-2-propoxy, the 3-bromo-1,1,1-trifluoro-2-propoxy, the 3-bromo-1,1,1-trifluoro-2-butoxy, the 4-bromo-3,3,4,4-tetrafluoro-1-butoxy, the chlorodifluoromethoxy, the 1,1,1,3,3,3-hexafluoro-2-propoxy, the 2-trifluoromethyl-2-propoxy, the 1,1,1-trifluoro-2-propoxy, the perfluoro-tert-butoxy, the 2,2,3,3,4,4,4-heptafluoro-1-butoxy, the 4,4,4-trifluoro-1-butoxy, the 2,2,3,3,3-pentafluoropropoxy, the perfluoroethoxy, the 1,2,2-bifluoroethoxy, in particular the 1,1,2,2-tetrafluoroethoxy, the 2,2,2-trifluoroethoxy, the trifluoromethoxy and preferably the difluoromethoxy group.

Mono- or di-1-4C-alkylamino represents an amino group, which is substituted by one or by two—identical or different—groups from the aforementioned 1-4C-alkyl groups. Examples which may be mentioned are the dimethylamino, the diethylamino and the diisopropylamino group.

1-4C-Alkylcarbonyl represents a group, which in addition to the carbonyl group contains one of the aforementioned 1-4C-alkyl groups. An example which may be mentioned is the acetyl group.

1-4C-Alkylcarbonylamino represents an amino group to which a 1-4C-alkylcarbonyl group is bonded. Examples which may be mentioned are the propionylamino (C₃H₇C(O)NH—) and the acetylamino group (acetamido group) (CH₃C(O)NH—).

1-4C-Alkoxycarbonylamino represents an amino group, which is substituted by one of the aforementioned 1-4C-alkoxycarbonyl groups. Examples, which may be mentioned, are the ethoxycarbonylamino and the methoxycarbonylamino group.

1-4C-Alkoxy-1-4C-alkoxycarbonyl represents a carbonyl group, to which one of the aforementioned 1-4C-alkoxy-1-4C-alkoxy groups is bonded. Examples which may be mentioned are the 2-(methoxy)ethoxycarbonyl (CH₃—O—CH₂CH₂—O—CO—) and the 2-(ethoxy)ethoxycarbonyl group (CH₃CH₂—O—CH₂CH₂—O—CO—).

1-4C-Alkoxy-1-4C-alkoxycarbonylamino represents an amino group, which is substituted by one of the aforementioned 1-4C-alkoxy-1-4C-alkoxycarbonyl groups. Examples which may be mentioned are the 2-(methoxy)ethoxycarbonylamino and the 2-(ethoxy)ethoxycarbonylamino group.

Hydroxypyrrolidino represents a pyrrolidino group, which is substituted by a hydroxy group. Examples which may be mentioned are the 2-hydroxypyrrolidino and the 3-hydroxypyrrolidino groups.

Hydroxyazetidino represents an azetidino group, which is substituted by a hydroxy group. An example which may be mentioned is the 3-hydroxyazetidino group.

Fluorazetidino represents an azetidino group, which is substituted by a fluoro atom. Examples which may be mentioned are the (2S)- and the (2R)-fluoroazetidino and in particular the 3-fluoroazetidino group.

N-1-4C-alkylpiperazino represents a piperazino group, in which one of the piperazino nitrogen atoms is substituted by one of the aforementioned 1-4C-alkyl groups. Examples which may be mentioned are the 4-methylpiperazino, the 4-ethylpiperazino and the 4-iso-propylpiperazino groups.

Possible salts of compounds of the formula 1—depending on substitution—are especially all acid addition salts. Particular mention may be made of the pharmacologically tolerable salts of the inorganic and organic acids customarily used in pharmacy. Those suitable are water-soluble and water-insoluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, malonic acid, fumaric acid, succinic acid, oxalic acid, tartaric acid, embonic acid, stearic acid, toluenesulfonic acid, methanesulfonic acid or 3-hydroxy-2-naphthoic acid, where the acids are used in salt preparation—depending on whether a mono- or polybasic acid is concerned and on which salt is desired—in an equimolar quantitative ratio or one differing therefrom.

Salts of the compounds of formula I according to the invention can be obtained by dissolving, the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or to which the desired acid is then added, if necessary upon heating. The acid can be employed in salt preparation, depending on whether a mono- or polybasic acid is concerned and depending on which salt is desired, in an equimolar quantitative ratio or one differing therefrom. The salts are obtained for example by evaporating the solvent or by precipitating upon cooling, by re-precipitating, or by precipitating with a non-solvent for the salt and separation, for example by filtration, of the salt after precipitation.

Pharmacologically intolerable salts, which can initially be obtained, for example, as process products in the production of the compounds according to the invention on the industrial scale, are converted into the pharmacologically tolerable salts by processes known to the person skilled in the art.

It is known to the person skilled in the art that the compounds according to the invention and their salts, if, for example, they are isolated in crystalline form, can contain various amounts of solvents. The invention therefore also comprises all solvates and in particular all hydrates of the compounds of the formula 1, and also all solvates and in particular all hydrates of the salts of the compounds of the formula 1.

The compounds of the formula 1 can have a center of chirality at the spiro carbon atom in 8-position of the basic skeleton. The occurrence of such a center of chirality depends on the nature and the position of the substituents R4 and R5. A center of chirality arises for example if R4 is different from R5. The invention thus relates to all feasible stereoisomers in any desired mixing ratio to another, including the pure stereoisomers, which are a preferred subject of the invention.

The invention therefore relates to all of the following stereoisomers of the formula 1:

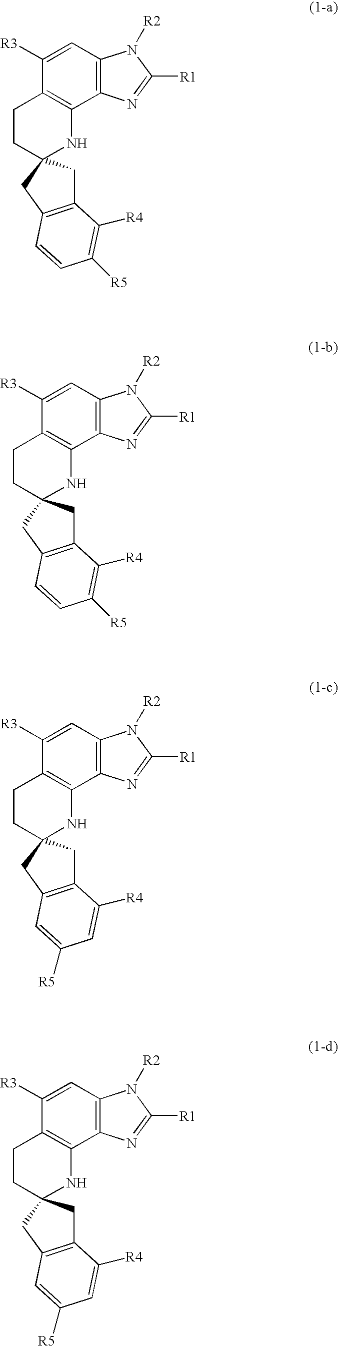

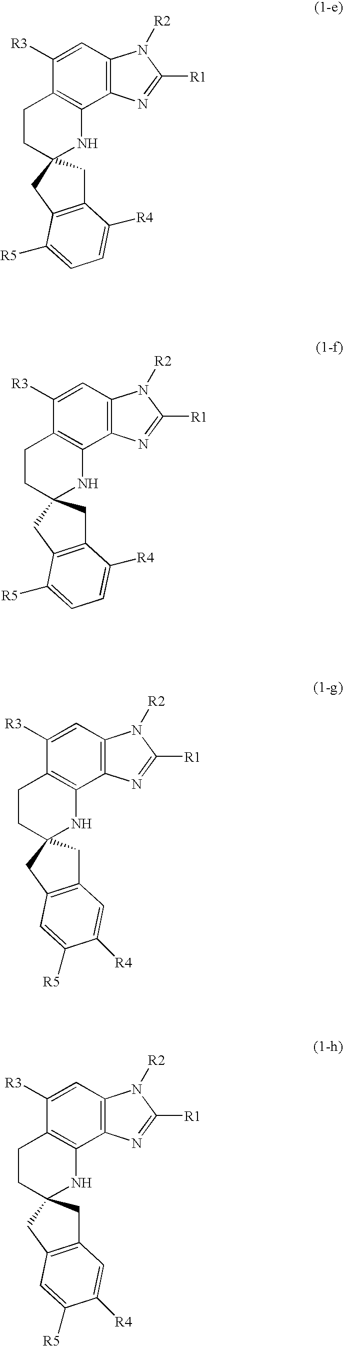

The pure stereoisomers of the compounds of the formula 1 and salts according to the present invention can be obtained e.g. by asymmetric synthesis, by using chiral starting compounds in synthesis and by splitting up stereoisomeric mixtures obtained in synthesis. Preferably, the pure stereoisomers of the compounds of the formula 1 are obtained by using chiral starting compounds.

Stereoisomeric mixtures of compounds of the formula 1 can be split up into the pure stereoisomers by methods known to a person skilled in the art. Preferably, the mixtures are separated by chromatography or (fractional) crystallization. For enantiomeric mixtures the split up is preferably done by forming diastereomeric salts by adding chiral additives like chiral acids, subsequent resolution of the salts and release of the desired compound from the salt. Alternatively, derivatization with chiral auxiliary reagents can be made, followed by diastereomer separation and removal of the chiral auxiliary group. Furthermore, enantiomeric mixtures can be separated using chiral separating columns in chromatography. Another suitable method for the separation of enantiomeric mixtures is the enzymatic separation.

One embodiment of the invention (embodiment 1) to be emphasized are the compounds of the formula 1, in which

R4 and R5 are each hydrogen,

and their salts.

Another embodiment of the invention (embodiment 2) to be emphasized are the compounds of the formula 1, in which

R1 is 1-4C-alkyl,

and their salts.

Another embodiment of the invention (embodiment 3) to be emphasized are the compounds of the formula 1, in which

R2 is hydrogen or 1-4C-alkyl,

and their salts.

Another embodiment of the invention (embodiment 4) to be emphasized are the compounds of the formula 1, in which

R3 is the group —CO—NR31R32,
where
R31 is hydrogen, hydroxyl, 1-7C-alkyl, 3-7C-cycloalkyl, hydroxy-1-4C-alkyl, 1-4C-alkoxy-1-4C-alkyl, 1-4C-alkylcarbonyl-1-4C-alkyl, 1-4C-alkylcarbonyl or 1-4C-alkoxycarbonyl and
R32 is hydrogen, 1-7C-alkyl, hydroxy-1-4C-alkyl, or 1-4C-alkoxy-1-4C-alkyl,
or where
R31 and R32 together, including the nitrogen atom to which both are bonded, are a pyrrolidino, hydroxypyrrolidino, piperidino, piperazino, azetidino, hydroxyazetidino, fluorazetidino, aziridino, N-1-4C-alkylpiperazino or morpholino group,
   and their salts.

An embodiment of the invention (embodiment 5) to be particularly emphasized are the compounds of the formula 1, in which

R1 is methyl,

and their salts.

Another embodiment of the invention (embodiment 6) to be particularly emphasized are the compounds of the formula 1, in which

R2 is hydrogen or methyl,

and their salts.

The invention also relates to compounds of the formula 1, in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 1-4C-alkoxy, 3-7C-cycloalkyl, 3-7C-cycloalkyl-1-4C-alkyl, hydroxy-1-4C-alkyl, 1-4C-alkoxy-1-4C-alkyl, 2-4C-alkenyl, 2-4C-alkynyl or fluoro-1-4C-alkyl,
R3 is hydrogen, halogen, fluoro-1-4C-alkyl, 1-4C-alkyl, 2-4C-alkenyl, 2-4C-alkynyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl, 1-4C-alkoxy-1-4C-alkyl or 1-4C-alkylcarbonyl-1-4C-alkyl, 1-4C-alkylcarbonyl or 1-4C-alkoxycarbonyl and
   R32 is hydrogen, 1-7C-alkyl, hydroxy-1-4C-alkyl, or 1-4C-alkoxy-1-4C-alkyl,
   or where
   R31 and R32 together, including the nitrogen atom to which both are bonded, are a pyrrolidino, hydroxypyrrolidino, piperidino, piperazino, azetidino, hydroxyazetidino, aziridino, N-1-4C-alkylpiperazino or morpholino group,
R4 and R5 are identical or different substituents selected from the group consisting of hydrogen, 1-4C-alkyl, hydroxy-1-4C-alkyl, 1-4C-alkoxy, 1-4C-alkoxy-1-4C-alkyl, hydroxy-1-4C-alkoxy, 1-4C-alkoxy-1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, trifluoromethyl, halo-1-4C-alkoxy, nitro, amino, mono- or di-1-4C-alkylamino, 1-4C-alkylcarbonylamino, 1-4C-alkoxycarbonylamino, 1-4C-alkoxy-1-4C-alkoxycarbonylamino or sulfonyl,
   and their salts.

Compounds of the formula 1 which are to be mentioned are those,

in which
R1 is hydrogen, 1-4C-alkyl, 3-7C-cycloalkyl, 3-7C-cycloalkyl-1-4C-alkyl, 1-4C-alkoxy-1-4C-alkyl or hydroxyl-1-4C-alkyl,
R2 is hydrogen, 1-4C-alkyl, 3-7C-cycloalkyl, hydroxy-1-4C-alkyl, 1-4C-alkoxy-1-4C-alkyl, 2-4C-alkenyl, 2-4C-alkynyl or fluoro-1-4C-alkyl,
R3 is hydrogen, halogen, fluoro-1-4C-alkyl, 1-4C-alkyl, 2-4C-alkenyl, 2-4C-alkynyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, 3-7C-cycloalkyl, hydroxy-1-4C-alkyl, 1-4C-alkoxy-1-4C-alkyl, 1-4C-alkylcarbonyl-1-4C-alkyl, 1-4C-alkylcarbonyl or 1-4C-alkoxycarbonyl and
   R32 is hydrogen, 1-7C-alkyl, hydroxy-1-4C-alkyl, or 1-4C-alkoxy-1-4C-alkyl,
   or where
   R31 and R32 together, including the nitrogen atom to which both are bonded, are a pyrrolidino, hydroxypyrrolidino, piperidino, piperazino, azetidino, hydroxyazetidino, fluorazetidino, aziridino, N-1-4C-alkylpiperazino or morpholino group,
R4 and R5 are identical or different substituents selected from the group consisting of hydrogen, 1-4C-alkyl or halogen,
   and their salts.

Compounds of the formula 1 which are also to be mentioned are those,

in which
R1 is hydrogen, 1-4C-alkyl, 3-7C-cycloalkyl, 3-7C-cycloalkyl-1-4C-alkyl, 1-4C-alkoxy-1-4C-alkyl or hydroxyl-1-4C-alkyl,
R2 is hydrogen, 1-4C-alkyl, 3-7C-cycloalkyl, hydroxy-1-4C-alkyl, 1-4C-alkoxy-1-4C-alkyl, 2-4C-alkenyl, 2-4C-alkynyl or fluoro-1-4C-alkyl,
R3 is hydrogen, halogen, fluoro-1-4C-alkyl, 1-4C-alkyl, 2-4C-alkenyl, 2-4C-alkynyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
   where
   R31 is hydrogen, hydroxyl, 1-7C-alkyl, hydroxy-1-4C-alkyl, 1-4C-alkoxy-1-4C-alkyl or 1-4C-alkylcarbonyl-1-4C-alkyl, 1-4C-alkylcarbonyl or 1-4C-alkoxycarbonyl and
   R32 is hydrogen, 1-7C-alkyl, hydroxy-1-4C-alkyl or 1-4C-alkoxy-1-4C-alkyl, or where
   R31 and R32 together, including the nitrogen atom to which both are bonded, are a pyrrolidino, hydroxypyrrolidino, piperidino, piperazino, azetidino, hydroxyazetidino, aziridino, N-1-4C-alkylpiperazino or morpholino group,
R4 and R5 are identical or different substituents selected from the group consisting of hydrogen, 1-4C-alkyl or halogen,
   and their salts.

Compounds of the formula 1 which are to be particularly mentioned are those,

in which
R1 is hydrogen, 1-4C-alkyl or hydroxy-1-4C-alkyl,
R2 is hydrogen, 1-4C-alkyl, hydroxy-1-4C-alkyl or 2-4C-alkenyl,
R3 is carboxyl, 1-4C-alkoxycarbonyl, hydroxy-1-4C-alkyl, 1-4C-alkoxy-1-4C-alkyl or the group —CO—NR31R32,
   where
   R31 is hydrogen, hydroxyl, 1-7C-alkyl, 3-7C-cycloalkyl, hydroxy-1-4C-alkyl, 1-4C-alkoxy-14C-alkyl or 1-4C-alkylcarbonyl-1-4C-alkyl and
   R32 is hydrogen, 1-7C-alkyl, hydroxy-1-4C-alkyl or 1-4C-alkoxy-1-4C-alkyl,
   or where
   R31 and R32 together, including the nitrogen atom to which both are bonded, are a pyrrolidino, hydroxypyrrolidino, piperidino, piperazino, azetidino, hydroxyazetidino, fluorazetidino, aziridino, N-1-4C-alkylpiperazino or morpholino group,
R4 and R5 are identical or different substituents selected from the group consisting of hydrogen, 1-4C-alkyl or halogen,
   and their salts.

Compounds of the formula 1 which are also to be particularly mentioned are those,

in which
R1 is hydrogen, 1-4C-alkyl or hydroxy-1-4C-alkyl,
R2 is hydrogen, 1-4C-alkyl or hydroxy-1-4C-alkyl,
R3 is carboxyl, 1-4C-alkoxycarbonyl, hydroxy-1-4C-alkyl, 1-4C-alkoxy-1-4C-alkyl or the group —CO—NR31R32,
   where
   R31 is hydrogen, hydroxyl, 1-7C-alkyl, hydroxy-1-4C-alkyl, 1-4C-alkoxy-1-4C-alkyl or 1-4C-alkylcarbonyl-1-4C-alkyl and
   R32 is hydrogen, 1-7C-alkyl, hydroxy-1-4C-alkyl or 1-4C-alkoxy-1-4C-alkyl, or where
   R31 and R32 together, including the nitrogen atom to which both are bonded, are a pyrrolidino, hydroxypyrrolidino, piperidino, piperazino, azetidino, hydroxyazetidino, aziridino, N-1-4C-alkylpiperazino or morpholino group,
R4 and R5 are identical or different substituents selected from the group consisting of hydrogen, 1-4C-alkyl or halogen,
   and their salts.

Compounds of the formula 1 which are to be emphasized are those,

in which

R1 is 1-4C-alkyl,

R2 is hydrogen, 1-4C-alkyl or 2-4C-alkenyl,

R3 is carboxyl, 1-4C-alkoxycarbonyl, hydroxy-1-4C-alkyl or the group —CO—NR31R32,
where
R31 is hydrogen, 1-7C-alkyl, 3-7C-cycloalkyl, hydroxy-1-4C-alkyl, 1-4C-alkoxy-1-4C-alkyl or 1-4C-alkylcarbonyl-1-4C-alkyl and
R32 is hydrogen or 1-7C-alkyl,
or where
R31 and R32 together, including the nitrogen atom to which both are bonded, are a pyrrolidino, azetidino, hydroxyazetidino, fluorazetidino or morpholino group,

R4 and R5 are each hydrogen,

and their salts.

Compounds of the formula 1 which are also to be emphasized are those,

in which

R1 is 1-4C-alkyl,

R2 is 1-4C-alkyl,

R3 is the group —CO—NR31R32,
where
R31 is hydrogen, 1-7C-alkyl, 3-7C-cycloalkyl, 1-4C-alkoxy-1-4C-alkyl or 1-4C-alkylcarbonyl-1-4C-alkyl and
R32 is hydrogen or 1-7C-alkyl,
or where
R31 and R32 together, including the nitrogen atom to which both are bonded, are a pyrrolidino, azetidino, fluorazefidino or morpholino group,

R4 and R5 are each hydrogen,

and their salts.

Compounds of the formula 1 which are also to be emphasized are those,

in which

R1 is 1-4C-alkyl,

R2 is 1-4C-alkyl,

R3 is carboxyl, 1-4C-alkoxycarbonyl or the group —CO—NR31R32,
where
R31 is hydrogen, hydroxy-1-4C-alkyl or 1-7C-alkyl,
R32 is hydrogen or 1-7C-alkyl and

R4 and R5 are each hydrogen,

and their salts.

Compounds of the formula 1 which are also to be particularly emphasized are those,

in which

R1 is methyl,

R2 is methyl,

R3 is the group —CO—NR31R32,
where
R31 is hydrogen, methyl, cyclopropyl, 2-methoxyethyl, 3-methoxypropyl or 2-oxo-propyl and
R32 is hydrogen or methyl,
or where
R31 and R32 together, including the nitrogen atom to which both are bonded, are a pyrrolidino, azetidino, 3-fluorazetidino or morpholino group,

R4 and R5 are each hydrogen,

and their salts.

Exemplary preferred compounds according to the invention are those compounds of the formula 1, wherein R1, R2, R3, R4 and R5 have the meanings as given in Table A (Me=CH₃. Et=C₂H₅), and the salts of these compounds. These compounds are either described by way of example as final products or can be prepared in an analogous manner using for example the process steps described below.

TABLE A

| R1 | R2 | R3 | R4 | R5 |
|---|---|---|---|---|
| Me | Me | CH₂OH | H | H |
| Me | Me | CH₂OCH₃ | H | H |
| Me | Me | C(O)NHMe | H | H |
| Me | Me | C(O)N-pyrrolidine | H | H |
| Me | Me | C(O)NH(CH₂)₂OH | H | H |
| Me | Me | C(O)NH(CH₂)₂OMe | H | H |
| Me | Me | C(O)NH₂ | H | H |
| Me | Me | C(O)N-morpholine | H | H |
| Me | Me | C(O)NMe₂ | H | H |
| Me | Me | C(O)N-aziridine | H | H |
| Me | Me | C(O)OEt | H | H |
| Me | Me | C(O)OH | H | H |
| Me | Me | C(O)N-azetidine | H | H |
| Me | Me | C(O)NH(CH₂)₂Me | H | H |
| Me | Me | C(O)NHCH₂CH(OH)CH₂OH | H | H |
| Me | Me | C(O)NH-cyclopropyl | H | H |
| Me | Me | H | H | H |
| Me | Me | C(O)NHEt | H | H |
| Me | Me | C(O)NH(CH₂)₃OH | H | H |
| Me | Me | C(O)NH(CH₂)₃OMe | H | H |
| Me | Me | C(O)NHCH₂C(O)CH₃ | H | H |
| Me | Me | C(O)N-3-fluorazetidine | H | H |
| Me | Me | C(O)N(CH₃)—(CH₂)₂OH | H | H |
| Me | Me | C(O)N(CH₃)—(CH₂)₂OMe | H | H |
| Me | Me | C(O)N(CH₃)—(CH₂)₃OH | H | H |
| Me | Me | C(O)N(CH₃)—(CH₂)₃OMe | H | H |
| Me | H | CH₂OH | H | H |
| Me | H | CH₂OCH₃ | H | H |
| Me | H | C(O)NHMe | H | H |
| Me | H | C(O)N-pyrrolidine | H | H |
| Me | H | C(O)NH(CH₂)₂OH | H | H |
| Me | H | C(O)NH(CH₂)₂OMe | H | H |
| Me | H | C(O)NH₂ | H | H |
| Me | H | C(O)N-morpholine | H | H |
| Me | H | C(O)NMe₂ | H | H |
| Me | H | C(O)N-aziridine | H | H |
| Me | H | C(O)OEt | H | H |
| Me | H | C(O)OH | H | H |
| Me | H | C(O)N-azetidine | H | H |
| Me | H | C(O)NH(CH₂)₂Me | H | H |
| Me | H | C(O)NHCH₂CH(OH)CH₂OH | H | H |
| Me | H | C(O)NH-cyclopropyl | H | H |
| Me | H | H | H | H |
| Me | H | C(O)NHEt | H | H |
| Me | H | C(O)NH(CH₂)₃OH | H | H |
| Me | H | C(O)NH(CH₂)₃OMe | H | H |
| Me | H | C(O)NHCH₂C(O)CH₃ | H | H |
| Me | H | C(O)N-3-fluorazetidine | H | H |
| Me | H | C(O)N(CH₃)—(CH₂)₂OH | H | H |
| Me | H | C(O)N(CH₃)—(CH₂)₂OMe | H | H |
| Me | H | C(O)N(CH₃)—(CH₂)₃OH | H | H |
| Me | H | C(O)N(CH₃)—(CH₂)₃OMe | H | H |

Exemplary particularly preferred compounds according to the invention are those described by way of example and the salts of these compounds.

The compounds according to the invention can be synthesized from corresponding starting compounds, for example according to the reaction schemes given below. The synthesis is carried out in a manner known to the expert, for example as described in more detail in the following examples.

As outlined in scheme 1, the compounds of the formula 1 can be obtained by reduction of the carbonyl group in the corresponding compounds of the formula 2 by methods which are familiar to a person skilled in the art, for example using triethylsilane/trifluoroacetic acid (West et al., J. Org. Chem. 1973, 38, 2675-2681) or, for example using lithium aluminium hydride in the case when R3 is a group which can not be reduced under these conditions like for example R3=hydrogen.

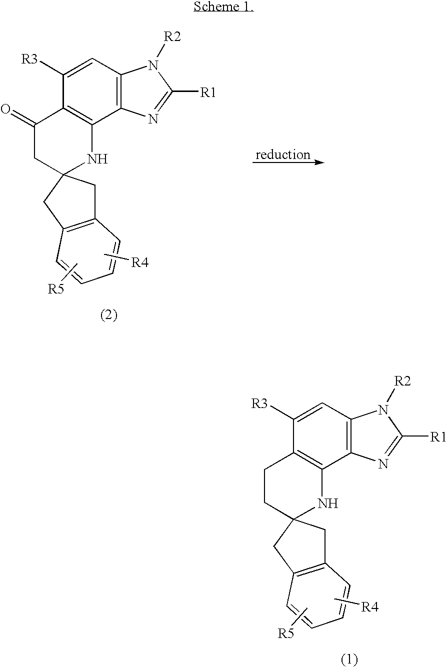

Compounds of the formula 2 can be prepared for example as outlined in scheme 2. In a first step ketones of the formula 3 are reacted with spiro-amino acid derivatives of the formula 4 (wherein Y is a suitable leaving group, for example an 1-4C-alkoxy group, e.g. an ethoxy group) to give compounds of the formula 5. In a second step, compounds of the formula 5 are oxidized by standard procedures using a suitable oxidizing agent (e.g. chloranil or 2,3-dichloro-5,6-dicyanobenzoquinone) to give compounds of the formula 2.

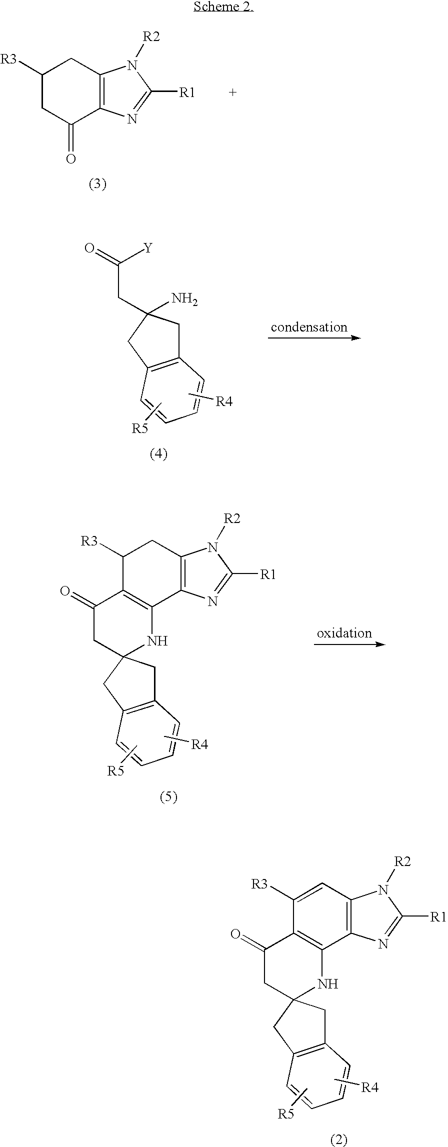

Ketones of the formula 3 can be prepared as shown, for example, in scheme 3 performing the cyclization reaction of compounds of the formula 7 in the presence of a primary amine (R2≠H) or ammonia (R2=H) under conditions known to the expert. The preparation of compounds of the formula 7 can be achieved by several methodologies known to the expert; two examples are illustrated in scheme 3. The reduction and subsequent acylation of azo-compounds of the formula 6 is performed in a manner known to the expert, for example as described by A. Treibs, R. Zinsmeister in Chem. Ber. 1957, 90, 87-92. Alternatively, aromatic compounds of the formula 8 can be reduced by strong reducing agents followed by an acidic workup, for example as described by Kuehne, Lambert in Org. Synth.; Coll. Vol. V, 1973, 400 or by A. Mann, C. Humblet in J. Med. Chem., 1985, 28, 1440-1446. Compounds of the formula 6 or 8 are known, for example from the patent FR2242984, or from Allan, Collect. Czech. Chem. Commun. 1966, 31, 4129, or they can be prepared using analogous process steps.

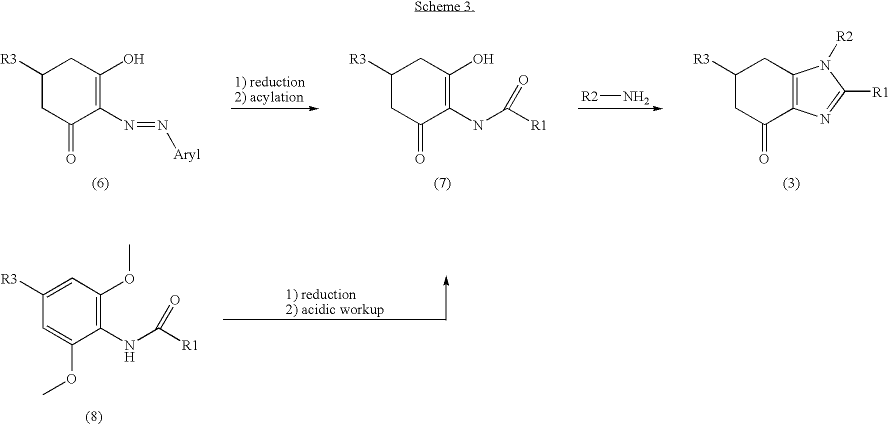

As outlined in scheme 4, the required β-amino acid derivatives of the general formula 4 can be prepared from the corresponding β-hydroxy acids of the formula 9, wherein Y is a suitable leaving group, for example an 1-4C-alkoxy group, e.g. an ethoxy group, by methods familiar to a person skilled in the art, like for example the Ritter reaction in analogy to the procedure described for example in Org. React. 1969, 17, 213. Compounds of the formula 9 are known to a person skilled in the art, for example from J. Chem. Soc. 1960, 4115, or they can be prepared using analogous process steps. If acetonitrile is used for the Ritter reaction, β-amino acids of the formula 4* with PG=acetyl are obtained. The protecting group PG in compounds of the formula 4* can then be cleaved from the amino functionality by methods known to the expert, for example if PG is an acetyl group, it can be cleaved by acidic hydrolysis to give compounds of the formula 4. The group Y can be transformed into any other group Y by standard procedures known to the expert, for example by esterification.

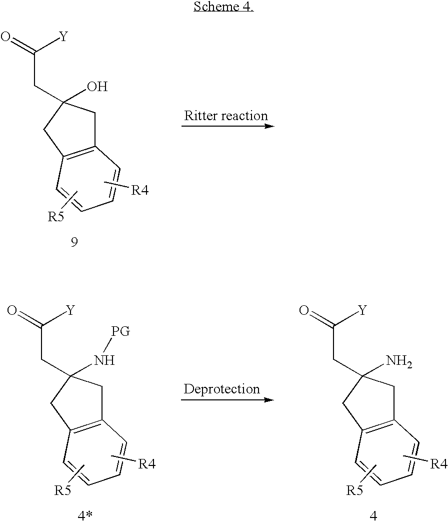

The derivatization, if any, of the compounds obtained according to the schemes above (e.g. conversion of a group R3 into another group R3 or conversion of a hydroxyl group into an alkoxy or ester group) is likewise carried out in a manner known per se. If, for example, compounds of the formula 1 where R3=—CO—NR31R32 are desired, an appropriate derivatization can be performed in a manner known per se (e.g. conversion of an ester or a carboxylic acid into an amide), preferably at the stage of compounds of the formula 1 or at the stage of any intermediate thereof.

The reaction steps outlined above are carried out in a manner known per se, e.g. as described in more detail in the examples.

The present invention further relates to compounds of the formula 2, 4 and 5 shown above, which are intermediates in the process of producing the compounds of the formula 1 according to the present invention. R1, R2, R3, R4, R5 are thereby defined as for compounds of the formula 1 and Y is a suitable leaving group, preferably a 1-4C-alkoxy group.

The person skilled in the art knows on the basis of his/her knowledge and on the basis of those synthesis routes, which are shown and described within the description of this invention, how to find other possible synthesis routes for compounds according to this invention. All synthesis routes described herein as well as all other possible synthesis routes are also part of this invention.

### ADVANTAGEOUS EFFECTS

The excellent gastric protective action and the gastric acid secretion-inhibiting action of the compounds according to the invention can be demonstrated in investigations on animal experimental models. The compounds of the formula 1 according to the invention investigated in the model mentioned below have been provided with numbers which correspond to the numbers of these compounds in the examples.

Testing of the Secretion-Inhibiting Action on the Perfused Rat Stomach

In Table A which follows, the influence of the compounds of the formula 1 according to the invention on the pentagastrin-stimulated acid secretion of the perfused rat stomach after intraduodenal administration in vivo is shown.

TABLE A

| | Dose | Inhibition of |
|---|---|---|
| | (μmol/kg) | acid secretion |
| No. | i.d. | (%) |
| 4 | 1 | >80 |
| 11 | 1 | >80 |
| 14 | 1 | >80 |
| 15 | 1 | >80 |
| 16 | 1 | >80 |
| 18 | 1 | >80 |
| 21 | 1 | >80 |
| 22 | 1 | >80 |
| 23 | 1 | >80 |
| 24 | 1 | >80 |
| 25 | 1 | >80 |
| 27 | 1 | >80 |

Methodology

The abdomen of anesthetized rats (CD rat, female, 200-250 g; 1.5 g/kg i.m. urethane) was opened after tracheotomy by a median upper abdominal incision and a PVC catheter was fixed transorally in the esophagus and another via the pylorus such that the ends of the tubes just projected into the gastric lumen. The catheter leading from the pylorus led outward into the right abdominal wall through a side opening.

After thorough rinsing (about 50-100 ml), warm (37° C.) physiological NaCl solution was conbinuously passed through the stomach (0.5 ml/min, pH 6.8-6.9; Braun-Unita I). The pH (pH meter 632, glass electrode EA 147; φ=5 mm, Metrohm) and, by titration with a freshly prepared 0.01N NaOH solution to pH 7 (Dosimat 665 Metrohm), the secreted HCl were determined in the effluent in each case collected at an interval of 15 minutes.

The gastric secretion was stimulated by continuous infusion of 1 μg/kg (=1.65 mllh) of i.v. pentagastrin (left femoral vein) about 30 min after the end of the operation (i.e. after determination of 2 preliminary fractions). The substances to be tested were administered intraduodenally in a 2.5 ml/kg liquid volume 60 min after the start of the continuous pentagastrin infusion. The body temperature of the animals was kept at a constant 37.8-38° C. by infrared irradiation and heat pads (automatic, stepless control by means of a rectal temperature sensor).

### MODE(S) FOR CARRYING OUT THE INVENTION

The following examples serve to illustrate the invention in greater detail without restricting it. Likewise, further compounds of the formula 1 whose preparation is not described explicitly can be prepared in an analogous manner or in a manner familiar per se to the person skilled in the art using customary process techniques. The abbreviation min stands for minute(s), h for hour(s) and m.p. for melting point.

I. Final Compounds of the Formula 1

### 1. Ethyl 2,3-dimethyl-1′,3,3′,6,7,9-hexahydrospiro[imidazo[4,5-h]quinoline-8,2′-indene]-5-carboxylate

To a solution of 0.3 g (0.77 mmol) ethyl 2,3-dimethyl-6-oxo-1′,3,3′,6,7,9-hexahydrospiro[imidazo[4,5-h]-quinoline-8,2′-indene]-5-carboxylate in 3 ml trifluoroacetic acid were added 1.1 ml (6.9 mmol) triethylsilane and the mixture was stirred overnight at ambient temperature. The reaction mixture was neutralized with saturated aqueous sodium hydrogen carbonate and extracted with dichloromethane. The organic layer was separated, dried over anhydrous magnesium sulphate and concentrated in vacuo. Purification of the residue by column chromatography (silica gel, ethyl acetate/petroleum ether/triethylamine 5:4:1) and crystallization from diethyl ether yielded 140 mg (48%) of the title compound as a colourless solid. m.p. 167-168° C.

### 2. Methyl 3-allyl-2-methyl-1′,3,3′,6,7,9-hexahydrospiro[imidazo[4,5-h]quinoline-8,2′-indene]-5-carboxylate

To a solution of 1.8 g (4.5 mmol) methyl 3-allyl-2-methyl-6-oxo-1′,3,3′,6,7,9-hexahydrospiro[imidazo[4,5-h]quinoline-8,2′-indene]-5-carboxylate in 21 ml trifluoroacetic acid were added 4.3 ml (27 mmol) triethylsilane and the mixture was stirred at 40° C. After 3 d, an additional amount of 0.5 ml triethylsilane was added and stirring was continued for 1 d. The reaction mixture was evaporated and the residue was partitioned between saturated aqueous sodium hydrogen carbonate and ethyl acetate. The organic layer was separated, dried over anhydrous magnesium sulphate and concentrated in vacuo. Purification of the residue by column chromatography (silica gel, ethyl acetate/petroleum ether/triethylamine 5:5:1) and crystallization from ethyl acetate/n-heptane yielded 1.1 g (62%) of the title compound as a colourless solid. m.p. 142-142.5° C.

### 3. Ethyl 3-allyl-2-methyl-1′,3,3′,6,7,9-hexahydrospiro[imidazo[4,5-h]quinoline-8,2′-indene]-5-carboxylate

To a solution of 3.1 g (7.5 mmol) ethyl 3-allyl-2-methyl-6-oxo-1′,3,3′,6,7,9-hexahydrospiro[imidazo[4,5-h]quinoline-8,2′-indene]-5-carboxylate in 35 ml trifluoroacetic acid were added 7.2 ml (45 mmol) triethylsilane and the mixture was stirred at 40° C. for 2 d. The reaction mixture was evaporated and the residue was partitioned between saturated aqueous sodium hydrogen carbonate and ethyl acetate. The organic layer was separated, dried over anhydrous magnesium sulphate and concentrated in vacuo. Purification of the residue by column chromatography (silica gel, ethyl acetate/petroleum ether/triethylamine 6:5:1) and crystallization from ethyl acetate/n-heptane yielded 1.2 g (40%) of the title compound as a colourless solid. m.p. 150° C.

### 4. N,2,3-Trimethyl-1′,3,3′,6,7,9-hexahydrospiro[imidazo[4,5-h]quinoline-8,2′-indene]-5-carboxamide

To a solution of 0.4 g (1.1 mmol) N,2,3-trimethyl-6-oxo-1′,3,3′,6,7,9-hexahydrospiro[imidazo[4,5-h]quinoline-8,2′-indene]-5-carboxamide in 10 ml trifluoroacetic acid was added triethylsilane [5×0.7 ml (6.9 mmol)] at 40° C. over a period of 12 d. The reaction mixture was neutralized with 1N aqueous sodium hydroxide and extracted with dichloromethane. The organic layer was separated, dried over anhydrous magnesium sulphate and concentrated in vacuo. Purification of the residue by column chromatography (silica gel, toluene/dioxane/methanol 6:3:1) and crystallization from diethyl ether yielded 160 mg (41%) of the title compound as a colourless solid. m.p. 237-238° C.

### 5. 2,3-Dimethyl-1′,3,3′,6,7,9-hexahydrospiro[imidazo[4,5-h]quinoline-8,2′-indene]-5-carboxylic acid

To a solution of 2.56 g (6.8 mmol) ethyl 2,3-dimethyl-1′,3,3′,6,7,9-hexahydrospiro[imidazo[4,5-h]quinoline-8,2′-indene]-5-carboxylate in 140 ml dioxane was added a solution of 1.63 g (68.1 mmol) lithium hydroxide in 55 ml water and the mixture was stirred overnight at 60° C. The reaction mixture was cooled down, neutralized with 6N hydrochloric acid and evaporated to dryness. The crude product (45 wt-%) was used without purification in the next step.

### 6. (2,3-Dimethyl-1′,3,3′,6,7,9-hexahydrospiro[imidazo[4,5-h]quinoline-8,2′-inden]-5-yl)-methanol

To a suspension of 1.17 g (3.12 mmol) ethyl 2,3-dimethyl-1′,3,3′,6,7,9-hexahydrospiro[imidazo[4,5-h]-quinoline-8,2′-indene]-5-carboxylate in 15 ml dried tetrahydrofuran were dropwise added 9.2 ml (9.2 mmol) diisobutylaluminum hydride (1M in toluene) at 0° C. After 1 h, an additional amount of 3 ml (3 mmol) diisobutylaluminum hydride was added and stirring was continued for 1.5 h. To the reaction mixture were added a few drops methanol and potassium sodium L-tartrate tetrahydrate. After 1 h, silica gel was added to the resulting emulsion and the mixture was evaporated to dryness and put on a column with silica gel. Elution with dichloromethane/methanol 13:1 and crystallization from ethyl acetate/diethyl ether yielded 0.25 g (24%) of the title compound as a colourless solid. m.p. 272-274° C.

### 7. 3-Allyl-N,2-dimethyl-1′,3,3′6,7,9-hexahydrospiro[imidazo[4,5-h]quinoline-8,2′-indene]-5-carboxamide

To a solution of 1.8 g (16 mmol) potassium tert-butoxide in 50 ml tert-butanol were added 0.8 g (2 mmol) ethyl 3-allyl-2-methyl-1′,3,3′,6,7,9-hexahydrospiro[imidazo[4,5-h]quinoline-8,2′-indene]-5-carboxylate and the mixture was stirred at 55° C. for 5 h. After stirring overnight at 40° C., the mixture was evaporated, neutralized with 2N hydrochloric acid and evaporated to dryness. To the residue were added 50 ml N,N-dimethylformamide and 2.57 g (8 mmol) O-(1H-benzotriazol-1-yl)-N,N,N′,N′-tetramethyl-uronium tetrafluoroborate (TBTU). At 0° C., 5 ml methylamine (7.5M in N,N-dimethylformamide) were added and the mixture was stirred 24 h at ambient temperature. The reaction mixture was evaporated to dryness and the residue was partitioned between saturated aqueous ammonium chloride and dichloromethane. The organic layer was separated, dried over anhydrous magnesium sulphate and concentrated in vacuo. Purification of the residue by column chromatography (silica gel, ethyl acetate/petroleum ether/triethylamine 5:5:1) and crystallization from ethyl acetate/n-heptane yielded 0.24 g (31%) of the title compound as a colourless solid. m.p. 226-227° C.

### 8. 3-Allyl-N,N,2-dimethyl-1′,3,3′,6,7,9-hexahydrospiro[imidazo[4,5-h]quinoline-8,2′-indene]-5-carboxamide

To a solution of 2.25 g (20 mmol) potassium tert-butoxide in 55 ml tert-butanol were added 0.97 g (2.5 mmol) methyl 3-allyl-2-methyl-1′,3,3′,6,7,9-hexahydrospiro[imidazo[4,5-h]quinoline-8,2′-indene]-5-carboxylate and the mixture was stirred at 55° C. After 50 min, 10 ml water were added, the mixture was neutralized with 2N hydrochloric acid and evaporated to dryness. To the residue were added 50 ml N,N-dimethylformamide and 3.21 g (10 mmol) O-(1H-benzotriazol-1-yl)-N,N,N′,N′-tetramethyl-uronium tetrafluoroborate (TBTU). At 0° C., 3 ml liquified dimethylamine were added and the mixture was stirred 3 d at ambient temperature. The reaction mixture was evaporated to dryness and the residue was partitioned between saturated aqueous ammonium chloride and dichloromethane. The organic layer was separated, dried over anhydrous magnesium sulphate and concentrated in vacuo. Purification of the residue by column chromatography (silica gel, ethyl acetate/triethylamine 9:1) and crystallization from ethyl acetate/n-heptane yielded 0.81 g (80%) of the title compound as a colourless solid. m.p. 172-173° C.

### 9. N,2-dimethyl-1′,3,3′,6,7,9-hexahydrospiro[imidazo[4,5-h]quinoline-8,2′-indene]-5-carboxamide

A mixture of 0.22 g (0.57 mmol) 3-allyl-N,2-dimethyl-1′,3,3′,6,7,9-hexahydrospiro[imidazo[4,5-h]quinoline-8,2′-indene]-5-carboxamide, 0.53 g (3.4 mmol) 1,3-dimethylbarbituric acid and 0.2 g (0.17 mmol) palladium tetrakis(triphenylphosphine) in 25 ml degassed dichloromethane was refluxed for 3 d under argon. The reaction mixture was partitioned between saturated aqueous sodium hydrogen carbonate and dichloromethane. The organic layer was separated, dried over anhydrous magnesium sulphate and concentrated in vacuo. Purification of the residue by column chromatography (silica gel, ethyl acetate/methanol/triethylamine 9:1:1), subsequent ion exchange chromatography (Dowex 50WX8, eluent 1N ammonia) and crystallization from ethyl acetate/diisopropyl ether yielded 31 mg (16%) of the title compound as a colourless solid. m.p. 240-241° C.

### 10. N,N,2-Trimethyl-1′,3,3′,6,7,9-hexahydrospiro[imidazo[4,5-h]quinoline-8,2′-indene]-5-carboxamide

A mixture of 0.4 g (1 mmol) 3-allyl-N,N,2-trimethyl-1′,3,3′,6,7,9-hexahydrospiro[imidazo[4,5-h]quinoline-8,2′-indene]-5-carboxamide, 0.47 g (3 mmol) 1,3-dimethylbarbituric acid and 0.25 g (0.22 mmol) palladium tetrakis(triphenylphosphine) in 50 ml degassed dichloromethane was refluxed for 5 d under argon. The reaction mixture was partitioned between saturated aqueous sodium hydrogen carbonate and dichloromethane. The organic layer was separated, dried over anhydrous magnesium sulphate and concentrated in vacuo. Purification of the residue by column chromatography (silica gel, ethyl acetate/methanol/triethylamine 9:1:1) delivered crude product, which was further purified by column chromatography (silica gel, dichloromethane/methanol 20:1). Crystallization from ethyl acetate/n-heptane yielded 89 mg (25%) of the title compound as a colourless solid. m.p. 183° C.

### 11. N,N,2,3-tetramethyl-1′,3,3′,6,7,9-hexahydrospiro[imidazo[4,5-h]quinoline-8,2′-indene]-5-carboxamide

To a suspension of 2.0 g (2.3 mmol, crude product) 2,3-dimethyl-1′,3,3′,6,7,9-hexahydrospiro[imidazo[4,5-h]quinoline-8,2′-indene]-5-carboxylic acid in 20 ml N,N-dimethylformamide were added 1.46 g (4.6 mmol) O-(1 benzotriazol-1-yl)-N,N,N′,N′-tetramethyl-uronium tetrafluoroborate (TBTU) and the mixture was heated to 40° C. After 30 min, 5.7 ml (11.4 mmol) dimethylamine (2M in tetrahydrofuran) were added at 0° C. After 30 min, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 9:1) and crystallized from diethyl ether to yield 0.72 g (85%) of the title compound as a colourless solid. m.p. 178-179° C.

### 12. N-(2-hydroxyethyl)-N,2,3-trimethyl-1′,3,3′,6,7,9-hexahydrospiro[imidazo[4,5-h]quinoline-8,2′-indene]-5-carboxamide

To a suspension of 2.0 g (2.3 mmol, crude product) 2,3-dimethyl-1′,3,3′,6,7,9-hexahydrospiro[imidazo[4,5-h]quinoline-8,2′-indene]-5-carboxylic acid in 20 ml N,N-dimethylformamide were added 1.46 g (4.6 mmol) O-(1 benzotriazol-1-yl)-N,N,N′,N′-tetramethyl-uronium tetrafluoroborate (TBTU) and the mixture was heated to 40° C. After 30 min, 0.85 g (11.3 mmol) 2-(methylamino)ethanol were added at 0° C. After 2 h, the reaction mixture was partitioned between water and dichloromethane. The organic layer was separated, dried over anhydrous magnesium sulphate and evaporated. The residue was purified by column chromatography (silica gel, ethyl acetate/triethylamine 4:1) and crystallized from ethyl acetate to yield 0.23 g (25%) of the title compound as a colourless solid. m.p. 194-195° C.

### 13. 1-[(2,3-dimethyl-1′,3,3′,6,7,9-hexahydrospiro[imidazo[4,5-h]quinoline-8,2′-inden]-5-yl)-carbonyl]azetidin-3-ol

To a suspension of 0.72 g (1.44 mmol, crude product) 2,3-dimethyl-1′,3,3′,6,7,9-hexahydrospiro[imidazo[4,5-h]quinoline-8,2′-indene]-5-carboxylic acid in 40 ml N,N-dimethylformamide were added 1.16 g (3.6 mmol) O-(1 benzotriazol-1-yl)-N,N,N′,N′-tetramethyl-uronium tetrafluoroborate (TBTU) and the mixture was heated to 40° C. After 30 min, 0.32 g (4.38 mmol) azetidin-3-ol were added at room temperature. After 1 h, the reaction mixture was evaporated to dryness and the residue was partitioned between water and dichloromethane. The organic layer was separated, dried over anhydrous magnesium sulphate and evaporated. The residue was purified by column chromatography (silica gel, toluene/dioxane/methanol 6:3:1) and crystallized from diethyl ether to yield 0.39 g (67%) of the title compound as a colourless solid. m.p. 171-172° C.

### 14. 2,3-dimethyl-N-(2-oxopropyl)-1′,3,3′,6,7,9-hexahydrospiro[imidazo[4,5-h]quinoline-8,2′-indene]-5-carboxamide

To a suspension of 0.72 g (1.44 mmol, crude product) 2,3-dimethyl-1′,3,3′,6,7,9-hexahydrospiro[imidazo[4,5-h]quinoline-8,2′-indene]-5-carboxylic acid in 70 ml N,N-dimethylformamide were added 1.16 g (3.6 mmol) O-(1H-benzotriazol-1-yl)-N,N,N′,N′-tetramethyl-uronium tetrafluoroborate (TBTU) and the mixture was heated to 40° C. After 30 min, 1 ml (7.2 mmol) triethylamine and 0.47 g (4.29 mmol) 2-aminopropanone hydrochloride were added at 0° C. After stirring overnight, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 9:1) and crystallized from diethyl ether to yield 0.36 g (62%) of the title compound as a colourless solid. m.p. 222-223° C.

### 15. 5-[(3-fluoroazetidin-1-yl)carbonyl]-2,3-dimethyl-1′,3,3′,6,7,9-hexahydrospiro[imidazo[4,5-h]quinoline-8,2′-indene]

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1H-benzotriazol-1-yl)-N,N,N′,N′-tetramethyl-uronium tetrafluoroborate (TBTU) and the mixture was heated to 40° C. After 30 min, 0.8 ml (6 mmol) triethylamine and 0.34 g (3.1 mmol) 3-fluoroazetidine hydrochloride were added at room temperature. After 2 h, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from diethyl ether to yield 0.66 g (77%) of the title compound as a colourless solid. m.p. 259-260° C.

### 16. N-(2-methoxyethyl)-2,3-dimethyl-1′,3,3′,6,7,9-hexahydrospiro[imidazo[4,5-h]quinoline-8,2′-indene]-5-carboxamide

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1H-benzotriazol-1-yl)-N,N,N′,N′-tetramethyl-uronium tetrafluoroborate (TBTU) and the mixture was heated to 40° C. After 30 min, 0.46 ml (5.3 mmol) 2-methoxyethylamine were added at 0° C. After 4 h at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from ethyl acetate/diethyl ether to yield 0.7 g (81%) of the title compound as a colourless solid. m.p. 186-187° C.

### 17. N-(2-hydroxyethyl)-2,3-dimethyl-1′,3,3′,6,7,9-hexahydrospiro[imidazo[4,5-h]quinoline-8,2′-indene]-5-carboxamide

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1 benzotriazol-1-yl)-N,N,N′,N′-tetramethyl-uronium tetrafluoroborate (TBTU) and the mixture was heated to 40° C. After 30 min, 0.47 ml (7.9 mmol) 2-hydroxyethylamine were added at 0° C. After stirring overnight at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, toluene/dioxane/methanol 6:3:1) and crystallized from diethyl ether to yield 0.46 g (39%) of the title compound as a colourless solid. m.p. 167-168° C.

### 18. 2,3-dimethyl-1′,3,3′,6,7,9-hexahydrospiro[imidazo[4,5-h]quinoline-8,2′-indene]-5-carboxamide

To a suspension of 0.37 g (1.06 mmol, crude product) 2,3-dimethyl-1′,3,3′,6,7,9-hexahydrospiro[imidazo[4,5-h]quinoline-8,2′-indene]-5-carboxylic acid in 50 ml N,N-dimethylformamide were added 0.85 g (2.7 mmol) O-(1 benzotriazol-1-yl)-N,N,N′,N′-tetramethyl-uronium tetrafluoroborate (TBTU) and the mixture was heated to 40° C. After 30 min, 0.56 ml (3.9 mmol) ammonia (7N in methanol) were added at room temperature. After 3 h, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from diethyl ether to yield 0.05 g (14%) of the title compound as a colourless solid. m.p. 272-273° C.

### 19. N-[(2S)-2-hydroxypropyl]-N,2,3-trimethyl-1′,3,3′,6,7,9-hexahydrospiro[imidazo[4,5-h]-quinoline-8,2′-indene]-5-carboxamide

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1H-benzotriazol-1-yl)-N,N,N′,N′-tetramethyl-uronium tetrafluoroborate (TBTU) and the mixture was heated to 40° C. After 30 min, 0.7 g (7.9 mmol) (S)-1-methylamino-propan-2-ol were added at 0° C. After 1 h at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from ethyl acetate to yield 0.05 g (14%) of the title compound as a colourless solid. m.p. 258-259° C.

### 20. N-[(2S)-2-hydroxypropyl]-2,3-dimethyl-1′,3,3′,6,7,9-hexahydrospiro[imidazo[4,5-h]-quinoline-8,2′-indene]-5-carboxamide

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1 benzotriazol-1-yl)-N,N,N′,N′-tetramethyl-uronium tetrafluoroborate (TBTU) and the mixture was heated to 40° C. After 30 min, 0.6 g (8 mmol) (S)-1-amino-propan-2-ol were added at 0° C. After 2 h at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from diethyl ether to yield 0.56 g (65%) of the title compound as a colourless solid. m.p. 224-225° C.

### 21. N-(2-methoxyethyl)-N,2,3-trimethyl-1′,3,3′,6,7,9-hexahydrospiro[imidazo[4,5-h]quinoline-8,2′-indene]-5-carboxamide

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1H-benzotriazol-1-yl)-N,N,N′,N′-tetramethyl-uronium tetrafluoroborate (TBTU) and the mixture was heated to 40° C. After 30 min, 0.7 g (7.9 mmol) (2-methoxyethyl)methylamine were added at 0° C. After 3 h, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from diethyl ether to yield 0.6 g (67%) of the title compound as a colourless solid. m.p. 160-161° C.

### 22. 2,3-dimethyl-5-(pyrrolidin-1-ylcarbonyl)-1′,3,3′,6,7,9-hexahydrospiro[imidazo[4,5-h]-quinoline-8,2′-indene]

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1 benzotriazol-1-yl)-N,N,N′,N′-tetramethyl-uronium tetrafluoroborate (TBTU) and the mixture was heated to 40° C. After 30 min, 0.62 ml (7.5 mmol) pyrrolidine were added at 0° C. After 4 h at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from diethyl ether to yield 0.73 g (86%) of the title compound as a colourless solid. m.p. 263-264° C.

### 23. 5-(azetidin-1-ylcarbonyl)-2,3-dimethyl-1′,3,3′,6,7,9-hexahydrospiro[imidazo[4,5-h]-quinoline-8,2′-indene]

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1 benzotriazol-1-yl)-N,N,N′,N′-tetramethyl-uronium tetrafluoroborate (TBTU) and the mixture was heated to 40° C. After 30 min, 0.5 ml (7.5 mmol) azetidine were added at 0° C. After 3 h at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from diethyl ether to yield 0.52 g (63%) of the title compound as a colourless solid. m.p. 222-223° C.

### 24. N-cyclopropyl-2,3-dimethyl-1′,3,3′,6,7,9-hexahydrospiro[imidazo[4,5-h]quinoline-8,2′-indene]-5-carboxamide

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1H-benzotriazol-1-yl)-N,N,N′,N′-tetramethyl-uronium tetrafluoroborate (TBTU) and the mixture was heated to 40° C. After 30 min, 0.43 g (7.5 mmol) cyclopropylamine were added at 0° C. After 2 h at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from diethyl ether to yield 0.65 g (79%) of the title compound as a colourless solid. m.p. 247-248° C.

### 25. 2,3-dimethyl-5-(morpholin-4-ylcarbonyl)-1′,3,3′,6,7,9-hexahydrospiro[imidazo[4,5-h]-quinoline-8,2′-indene]

To a suspension of 0.74 g (2.13 mmol, crude product) 2,3-dimethyl-1′,3,3′,6,7,9-hexahydrospiro[imidazo[4,5-h]quinoline-8,2′-indene]-5-carboxylic acid in 100 ml N,N-dimethylformamide were added 1.71 g (5.3 mmol) O-(1 benzotriazol-1-yl)-N,N,N′,N′-tetramethyl-uronium tetrafluoroborate (TBTU) and the mixture was heated to 40° C. After 30 min, 0.65 ml (7.5 mmol) morpholine were added at 0° C. After stirring overnight at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, ethyl acetate/triethylamine 4:1) and crystallized from diethyl ether to yield 0.72 g (81%) of the title compound as a colourless solid. m.p. 284-285° C.

### 26. N-(3-hydroxypropyl)-2,3-dimethyl-1′,3,3′,6,7,9-hexahydrospiro[imidazo[4,5-h]quinoline-8,2′-indene]-5-carboxamide

To a suspension of 1.0 g (2.88 mmol, crude product) 2,3-dimethyl-1′,3,3′,6,7,9-hexahydrospiro[imidazo[4,5-h]quinoline-8,2′-indene]-5-carboxylic acid in 15 ml N,N-dimethylformamide were added 2.3 g (7.2 mmol) O-(1H-benzotriazol-1-yl)-N,N,N′,N′-tetramethyl-uronium tetrafluoroborate (TBTU) and the mixture was heated to 40° C. After 1 h, 0.44 ml (5.7 mmol) 3-amino-propan-1-ol were added and stirring was continued overnight at room temperature. The reaction mixture was evaporated to dryness and the residue was partitioned between water and dichloromethane. The organic layer was separated, dried over anhydrous magnesium sulphate and evaporated. The residue was crystallized from dichloromethane/ethyl acetate/triethylamine to yield 0.31 g (27%) of the title compound as a colourless solid. m.p. 237-238° C.

### 27. N-(3-methoxypropyl)-2,3-dimethyl-1′,3,3′,6,7,9-hexahydrospiro[imidazo[4,5-h]quinoline-8,2′-indene]-5-carboxamide

To a suspension of 1.0 g (2.88 mmol, crude product) 2,3-dimethyl-1′,3,3′,6,7,9-hexahydrospiro[imidazo[4,5-h]quinoline-8,2′-indene]-5-carboxylic acid in 5 ml tetrahydrofuran were added 0.93 g (5.7 mmol) N,N′-carbonyldiimidazole O-(1H-benzotriazol-1-yl)-N,N,N′,N′-tetramethyl-uronium tetrafluoroborate (TBTU) and the mixture was heated to 70° C. After 2 h, 1.2 ml (11.2 mmol) 3-methoxy-propylamine were added at 40° C. and stirring was continued for 1 h. The reaction mixture was partitioned between water and dichloromethane. The organic layer was separated, dried over anhydrous magnesium sulphate and evaporated. The residue was purified by column chromatography (silica gel, ethyl acetate/triethylamine 4:1) and crystallized from ethyl acetate/diethyl ether to yield 0.42 g (35%) of the title compound as a colourless solid. m.p. 188-189° C.

### 28. N,2,3-Trimethyl-1′,3,3′,6,7,9-hexahydrospiro[imidazo[4,5-h]quinoline-8,2′-indene]-5-carboxamide methanesulphonate

A suspension of 7.0 g (19.4 mmol) N,2,3-trimethyl-1′,3,3′,6,7,9-hexahydrospiro[imidazo[4,5-h]quinoline-8,2′-indene]5-carboxamide and 2.05 g (20.8 mmol) methanesulphonic acid were suspended in 80 ml methanol and the mixture was heated to 70° C. After 40 min, the solution was allowed to cool to room temperature. After 1 h at 0° C., the precipitate was collected, washed with water and dried to yield 5.7 g (65%) of the title compound as a colourless solid. m.p. 321-324° C.

### 29. N,2,3-Trimethyl-1′,3,3′,6,7,9-hexahydrospiro[imidazo[4,5-h]quinoline-8,2′-indene]-5-carboxamide hydrochloride

A suspension of 10.0 g (27.8 mmol) N,2,3-trimethyl-1′,3,3′,6,7,9-hexahydrospiro[imidazo[4,5-h]quinoline-8,2′-indene]-5-carboxamide in 100 ml methanol was heated to reflux. To the mixture were added 3 ml conc. hydrochloric acid and the clear solution was allowed to cool to room temperature. The resulting precipitate was collected, washed with methanol and dried to yield 5.7 g (52%) of the title compound as a colourless solid. m.p. 309-311° C.

### 30. N,2,3-Trimethyl-1′,3,3′,6,7,9-hexahydrospiro[imidazo[4,5-h]quinoline-8,2′-indene]-5-carboxamide malonate

A suspension of 7.0 g (19.4 mmol) N,2,3-trimethyl-1′,3,3′,6,7,9-hexahydrospiro[imidazo[4,5-h]quinoline-8,2′-indene]-5-carboxamide and 2.2 g (20.8 mmol) malonic acid were suspended in 80 ml methanol and the mixture was heated to 70° C. After 30 min, the solution was allowed to cool to room temperature. After 1 h at 0° C., the precipitate was collected, washed with water and dried to yield 6.95 g (77%) of the title compound as a colourless solid. m.p. 261-263° C.

### 31. N-(2-ethoxyethyl)-2,3-dimethyl-1′,3,3′,6,7,9-hexahydrospiro[imidazo[4,5-h]quinoline-8,2′-indene]-5-carboxamide

To a suspension of 1.0 g (2.9 mmol, crude product) 2,3-dimethyl-1′,3,3′,6,7,9-hexahydrospiro[imidazo[4,5-h]quinoline-8,2′-indene]-5-carboxylic acid in 15 ml N,N-dimethylformamide were added 2.3 g (7.2 mmol) O-(1 benzotriazol-1-yl)-N,N,N′,N′-tetramethyl-uronium tetrafluoroborate (TBTU) and the mixture was heated to 40° C. After 1 h, 0.5 g (5.2 mmol) 2-ethoxyethylamine were added at room temperature. After stirring overnight at room temperature, the reaction mixture was evaporated to dryness and the residue was partitioned between water and dichloromethane. The organic layer was separated, dried over anhydrous magnesium sulphate and evaporated. The residue was purified by column chromatography (silica gel, dichloromethane/methanol 13:1) and crystallized from ethyl acetate to yield 0.17 g (14%) of the title compound as a colourless solid. m.p. 180-181° C.

### 32. N-ethyl-2,3-dimethyl-1′,3,3′,6,7,9-hexahydrospiro[imidazo[4,5-h]quinoline-8,2′-indene]-5-carboxamide

To a suspension of 0.5 g (1.44 mmol, crude product) 2,3-dimethyl-1′,3,3′,6,7,9-hexahydrospiro[imidazo[4,5-h]quinoline-8,2′-indene]-5-carboxylic acid in 5 ml tetrahydrofuran were added 0.47 g (2.9 mmol) N,N′-carbonyldiimidazole and the mixture was heated to 70° C. After 4 h, 2.9 ml (5.8 mmol) ethylamine (2M in tetrahydrofuran) were added at room temperature. After stirring 2 d at room temperature, the reaction mixture was partitioned between water and dichloromethane. The organic layer was separated, dried over anhydrous magnesium sulphate and evaporated. The residue was crystallized from ethyl acetate/acetone/n-heptane to yield 0.23 g (43%) of the title compound as a colourless solid. m.p. 223-224° C.

### 33. N-(2-methoxyethyl)-2,3-dimethyl-1′,3,3′,6,7,9-hexahydrospiro[imidazo[4,5-h]quinoline-8,2′-indene]-5-carboxamide maleate

A suspension of 0.5 g (1.24 mmol) N-(2-methoxyethyl)-2,3-dimethyl-1′,3,3′,6,7,9-hexahydrospiro[imidazo[4,5-h]quinoline-8,2′-indene]-5-carboxamide and 0.16 g (1.38 mmol) maleic acid were suspended in 5 ml methanol and 2 ml 1,2-dichloroethane and the mixture was heated to 80° C. After complete solution, the mixture was allowed to cool to room temperature. The precipitate was collected, washed with acetone and dried to yield 0.44 g (68%) of the title compound as a colourless solid. m.p. 191-192° C.

### 34. N-(2-methoxyethyl)-2,3-dimethyl-1′,3,3′,6,7,9-hexahydrospiro[imidazo[4,5-h]quinoline-8,2′-indene]-5-carboxamide malonate

A suspension of 0.5 g (1.24 mmol) N-(2-methoxyethyl)-2,3-dimethyl-1′,3,3′,6,7,9-hexahydrospiro[imidazo[4,5-h]quinoline-8,2′-indene]-5-carboxamide and 0.14 g (1.35 mmol) malonic acid were suspended in 5 ml methanol and the mixture was heated to 40° C. After complete solution, the mixture was allowed to cool to room temperature. The precipitate was collected, washed with acetone and dried to yield 0.41 g (65%) of the title compound as a colourless solid. m.p. 176-177° C.

### 35. N-(2-methoxyethyl)-2,3-dimethyl-1′,3,3′,6,7,9-hexahydrospiro[imidazo[4,5-h]quinoline-8,2′-indene]-5-carboxamide hydrochloride

A solution of 0.5 g (1.24 mmol) N-(2-methoxyethyl)-2,3-dimethyl-1′,3,3′,6,7,9-hexahydrospiro[imidazo[4,5-h]quinoline-8,2′-indene]-5-carboxamide and 0.11 ml (1.36 mmol) conc. hydrochloric acid in 5 ml methanol was stirred at room temperature for 2 h. The precipitate was collected, washed with acetone and dried to yield 0.16 g (29%) of the title compound as a colourless solid. m.p. 294-295° C.

### 36. N-cyclopropyl-2,3-dimethyl-1′,3,3′,6,7,9-hexahydrospiro[imidazo[4,5-h]quinoline-8,2′-indene]-5-carboxamide oxalate

A solution of 0.1 g (0.26 mmol) N-cyclopropyl-2,3-dimethyl-1′,3,3′,6,7,9-hexahydrospiro[imidazo[4,5-h]-quinoline-8,2′-indene]-5-carboxamide and 23 mg (0.26 mmol) oxalic acid in 2 ml acetone/methanol was stirred at room temperature for 3 h. The precipitate was collected, washed with acetone and dried to yield 0.08 g (65%) of the title compound as a colourless solid. m.p. 242-243° C.

### 37. N-cyclopropyl-2,3-dimethyl-1′,3,3′,6,7,9-hexahydrospiro[imidazo[4,5-h]quinoline-8,2′-indene]-5-carboxamide methanesulphonate

To a solution of 0.3 g (0.78 mmol) N-cyclopropyl-2,3-dimethyl-1′,3,3′,6,7,9-hexahydrospiro[imidazo[4,5-h]quinoline-8,2′-indene]-5-carboxamide in 1 ml methanol were added 82 mg (0.85 mmol) methanesulphonic acid and the mixture was stirred at room temperature for 15 min. The precipitate was collected, washed with acetone and dried to yield 0.06 g (16%) of the title compound as a colourless solid. m.p. 308-309° C.

II. Starting Compounds

### A. 3-Hydroxy-5-oxocyclohex-3-ene-1-carboxylic acid

50.0 g (0.32 mol) 3,5-dihydroxybenzoic acid and 10.0 g Raney-Nickel were suspended in 175 ml 4N aqueous sodium hydroxide and hydrogenated for 5 d (60° C., 100-150 bar hydrogen). The mixture was filtered through Celite and the filtrate was acidified with conc. hydrochloric acid at 0° C. The resulting precipitate was collected, washed with ice-cold water and dried to give 25.2 g (50%) of the title compound. B.

### B. Methyl 3-hydroxy-5-oxo-4-[phenyldiazenyl]cyclohex-3-ene-1-carboxylate

To a solution of 8.64 g (0.216 mol) sodium hydride (60%) in 320 ml methanol were added 16.8 g (0.108 mol) 3-hydroxy-5-oxocyclohex-3-ene-1-carboxylic acid at 0° C. The mixture was stirred for 3 min and cooled to −10° C. To this solution was added dropwise a solution of benzenediazonium chloride in water [prepared by diazotization of 14.0 g (0.108 mol) aniline hydrochloride with 7.5 g (0.108 mol) sodium nitrite and 18 ml conc. hydrochloric acid] at 0° C. After 15 min stirring, the precipitate was collected, washed with water and suspended in 430 ml toluene and 75 ml methanol. 1.8 g p-toluenesulphonic acid monohydrate were added added and the mixture was refluxed with a Dean-Stark trap to remove water. After 22 h, the mixture was cooled down, washed with 200 ml saturated aqueous sodium hydrogen carbonate and 150 ml water. The organic layer was dried over anhydrous magnesium sulphate and concentrated in vacuo. Purification by column chromatography (aluminium oxide, ethyl acetate) and crystallization from ethyl acetate/n-heptane yielded 21.5 g (78%) of the title compound as a yellow solid. m.p. 117-118° C.

### C. Ethyl 3-hydroxy-5-oxo-4-[4-bromo-phenyldiazenyl]cyclohex-3-ene-1-carboxylate

To a solution of 11.25 g (0.49 mol) sodium in 200 ml ethanol were added dropwise 100 ml (0.47 mol) 2-(2-oxo-propyl)-succinic acid diethyl ester and the reaction mixture was refluxed for 2 h. The mixture was cooled down and evaporated to give an oil which was dissolved in 100 ml ethanol and 800 ml water. To this solution was added dropwise a suspension of 4-bromobenzenediazonium chloride in water [prepared by diazotization of 40 g (0.23 mol) 4-bromoaniline with 18.9 g (0.27 mol) sodium nitrite and hydrochloric acid] at 0° C. After 20 min stirring, the precipitate was collected, washed with water and dried in vacuo at 60° C. to give 71.8 g (84%) of the title compound. m.p. 169-171° C.

### D. Methyl 4-acetamido-3-hydroxy-5-oxocyclohex-3-ene-1-carboxylate

To a solution of 20.0 g (73 mmol) methyl 3-hydroxy-5-oxo-4-[phenyldiazenyl]cyclohex-3-ene-1-carboxylate in 42 ml acetic anhydride and 290 ml glacial acetic acid were added 28.8 g (0.44 mol) zinc powder portionwise at room temperature. After 40 min, the reaction mixture was filtered through Celite and the filter cake was washed with dioxane. The filtrate was concentrated in vacuo and coevaporated twice with toluene. The residue was purified by column chromatography using first ethyl acetate/triethylamine 9:1, then ethyl acetate/acetic acid 100:1 and ethyl acetate as eluent. Crystallization of the residue from ethyl acetate/n-heptane yielded 14.1 g (85%) of the title compound as a colourless solid. m.p. 131-132° C.

### E. Ethyl 4-acetamido-3-hydroxy-5-oxocyclohex-3-ene-1-carboxylate

73.0 g (198 mmol) ethyl 3-hydroxy-5-oxo-4-[4-bromo-phenyldiazenyl]cyclohex-3-ene-1-carboxylate were suspended in a mixture of 113 ml acetic anhydride and 730 ml glacial acetic acid. 78 g (1.193 mol) zinc powder were added portionwise at room temperature. The reaction mixture was stirred for 4 h, diluted with dioxane, filtered through a pad of silica gel, and concentrated. The residue was treated with dichloromethane, the precipitate (4-bromoacetanilide) was filtered off, and the filtrate was concentrated. The residue was purified by column chromatography (silica gel, toluene/dioxane 7:3) and crystallized from diisopropyl ether to yield 40.0 g (84%) of the title compound as white crystals. m.p. 111.5-113.5° C.

### F. Methyl 1-allyl-2-methyl-4-oxo-4,5,6,7-tetrahydro-1H-benzimidazole-6-carboxylate

A mixture of 0.91 g (4 mmol) methyl 4-acetamido-3-hydroxy-5-oxocyclohex-3-ene-1-carboxylate and 0.32 ml (4.2 mmol) allylamine in 4 ml toluene and 0.4 ml glacial acetic acid was heated to 150° C. for 40 min using microwave irradiation. The samples of 12 such runs were combined and evaporated to dryness. Purification of the residue by column chromatography (silica gel, toluene/dioxane/methanol 6:1:1) yielded 8.4 g (60%) of the title compound as a yellow oil. ¹H-NMR (CDCl₃), δ (ppm): 2.39 (s, 3H, CH₃), 2.79 (t, 2H), 3.02 (t, 2H), 3.21-3.35 (m, 1H), 3.72 (s, 3H, OCH₃), 4.47 (m, 2H), 4.90 (d, 1H), 5.27 (d, 1H), 5.80-5.99 (m, 1H).

### G. Ethyl 1-allyl-2-methyl-4-oxo-4,5,6,7-tetrahydro-1H-benzimidazole-6-carboxylate

A mixture of 0.97 g (4 mmol) ethyl 4-acetamido-3-hydroxy-5-oxocyclohex-3-ene-1-carboxylate and 0.32 ml (4.2 mmol) allylamine in 4 ml toluene and 0.4 ml glacial acetic acid was heated to 150° C. for 40 min using microwave irradiation. The samples of 20 such runs were combined and evaporated to dryness. Purification of the residue by column chromatography (silica gel, toluene/dioxane/methanol 6:1:1) and crystallization from ethyl acetate/n-heptane yielded 11.1 g (57%) of the title compound as a solid. m.p. 95-97° C.

### H. Ethyl 1,2-dimethyl-4-oxo-4,5,6,7-tetrahydro-1H-benzimidazole-6-carboxylate

To a solution of 30.0 g (124 mmol) ethyl 4-acetamido-3-hydroxy-5-oxocyclohex-3-ene-1-carboxylate in 250 ml toluene were added 75 ml (150 mmol) methylamine (2M in tetrahydrofuran) and 30 ml glacial acetic acid. The reaction mixture was transferred to an autoclave and heated for 2 h at 180° C. After cooling down, the solvent was removed and the residue was purified by column chromatography (silica gel, dichloromethane/methanol 9:1). Crystallization from diisopropyl ether yielded 25.0 g (85%) of the title compound as white crystals. m.p. 147.5-150.0° C.

### I. N,1,2-Trimethyl-4-oxo-4,5,6,7-tetrahydro-1H-benzimidazole-6-carboxamide

23.0 g (97.4 mmol) ethyl 1,2-dimethyl-4-oxo-4,5,6,7-tetrahydro-1H-benzimidazole-6-carboxylate were dissolved in 160 ml methylamine (40% in water), transferred to an autoclave, and heated to 150° C. for 3.5 h. After cooling down, the volatiles were removed to leave a dark oil. Purification by column chromatography (silica gel, toluene/dioxane/methanol 3:1:1) and crystallization from diisopropyl ether yielded 15.5 g (73%) of the title compound as white crystals. m.p. 194-196° C.

### J. Methyl 3-allyl-2-methyl-6-oxo-1′,3,3′,4,5,6,7,9-octahydrospiro[imidazo[4,5-h]quinoline-8,2′-indene]-5-carboxylate

A mixture of 4.4 g (20 mmol) ethyl (2-amino-2,3-dihydro-1H-inden-2-yl)acetate [liberated from its hydrochloride salt, ethyl (2-amino-2,3-dihydro-1H-inden-2-yl)acetate hydrochloride, example U, by treatment with triethylamine], 4.0 g (16 mmol) methyl 1-allyl-2-methyl-4-oxo-4,5,6,7-tetrahydro-1H-benzimidazole-6-carboxylate and 50 mg of p-toluenesulphonic acid monohydrate in xylene was heated under reflux with a Dean-Stark trap for 3 d. The solvent was removed and the residue was purified by column chromatography (silica gel, ethyl acetate/petroleum ether 9:1) to yield 2.41 g (92% purity) of the title compound as a solid. ¹H-NMR (CDCl₃), δ (ppm): 2.32 (s, 3H, CH₃), 2.64-2.90 (m, 3H), 3.18-3.32 (m, 4H), 3.65 (s, 3H, OCH₃), 4.27 (dd, 1H), 4.45 (m, 2H), 4.92 (d, 1H), 5.25 (d, 1H), 5.78-5.96 (m, 1H), 6.00 (bs, 1H, NH), 7.10-7.25 (m, 4H).

### K. Ethyl 3-allyl-2-methyl-6-oxo-1′,3,3′,4,5,6,7,9-octahydrospiro[imidazo[4,5-h]quinoline-8,2′-indene]-5-carboxylate

A mixture of 11.1 g (50.6 mmol) ethyl (2-amino-2,3-dihydro-1H-inden-2-yl)acetate [liberated from its hydrochloride salt, ethyl (2-amino-2,3-dihydro-1H-inden-2-yl)acetate hydrochloride, example U, by treatment with triethylamine], 11.1 g (42.3 mmol) ethyl 1-allyl-2-methyl-4-oxo-4,5,6,7-tetrahydro-1H-benzimidazole-6-carboxylate and 150 mg of p-toluenesulphonic acid monohydrate in xylene was heated under reflux with a Dean-Stark trap. After 3 d, an additional amount of 5.95 g (27 mmol) ethyl (2-amino-2,3-dihydro-1H-inden-2-yl)acetate was added and heating was continued for 1d. The solvent was removed and the residue was purified by column chromatography (silica gel, toluene/dioxane/methanol 6:3.5:0.5). Crystallization from ethyl acetate/n-heptane yielded 3.73 g (21%) of the title compound as a solid. m.p. 164-165° C.

### L. Ethyl 2,3-dimethyl-6-oxo-1′,3,3′,4,5,6,7,9-octahydrospiro[imidazo[4,5-h]quinoline-8,2′-indene]-5-carboxylate

A mixture of 4.48 g (23.4 mmol) ethyl (2-amino-2,3-dihydro-1H-inden-2-yl)acetate [liberated from its hydrochloride salt, ethyl (2-amino-2,3-dihydro-1H-inden-2-yl)acetate hydrochloride, example U, by treatment with triethylamine], 5.5 g (23.4 mmol) ethyl 1,2-dimethyl-4-oxo-4,5,6,7-tetrahydro-1H-benzimidazole-6-carboxylate and a catalytic amount of p-toluenesulphonic acid monohydrate in xylene was heated under reflux for 3 d. The solvent was removed and the residue was purified by column chromatography (silica gel, dichloromethane/methanol 99:1) to yield 1.97 g (21%) of the title compound as an oil. ¹H-NMR (CDCl₃): 1.21 (t, J=6.9 Hz, 3H), 2.30 (s, 3H), 2.58-2.64 (m, 1H), 2.78-2.85 (m, 2H), 3.13 (s, 2H), 3.19 (s, 2H), 3.26-3.32 (m, 1H), 3.45 (s, 3H), 4.07 (q, J=6.9 Hz, 2H), 4.22 (m, 1H), 6.00 (s, 1H, NH), 7.11-7.17 (m, 4H).

### M. N,2,3-Trimethyl-6-oxo-1′,3,3′,4,5,6,7,9-octahydrospiro[imidazo[4,5-h]quinoline-8,2′-indene]-5-carboxamide

A mixture of 3.6 g (16.4 mmol) ethyl (2-amino-2,3-dihydro-1H-inden-2-yl)acetate, [liberated from its hydrochloride salt, ethyl (2-amino-2,3-dihydro-1H-inden-2-yl)acetate hydrochloride, example U, by treatment with triethylamine], 4.3 g (18.2 mmol) N,1,2-trimethyl-4-oxo-4,5,6,7-tetrahydro-1H-benzimidazole-6-carboxamide and a catalytic amount of p-toluenesulphonic acid monohydrate in xylene was heated under reflux for 6 d. The solvent was removed and the residue was purified by column chromatography (silica gel, dichloromethane/methanol 99:1). Crystallization from diethyl ether yielded 1.3 g (55%) of the title compound. m.p. 93-94° C.

### N. Methyl 3-allyl-2-methyl-6-oxo-1′,3,3′,6,7,9-hexahydrospiro[imidazo[4,5-h]quinoline-8,2′-indene]-5-carboxylate

To a suspension of 1.98 g (4.9 mmol) methyl 3-allyl-2-methyl-6-oxo-1′,3,3′,4,5,6,7,9-octahydrospiro-[imidazo[4,5-h]quinoline-8,2′-indene]-5-carboxylate in 75 ml ethyl acetate were added 1.14 g (5.0 mmol) 2,3-dichloro-5,6-dicyanobenzoquinone. After 30 min, saturated aqueous sodium hydrogen carbonate was added and the mixture was extracted with ethyl acetate. The organic layer was separated, dried over anhydrous sodium sulphate and concentrated in vacuo. Purification of the residue by column chromatography (silica gel, toluene/dioxane/methanol 6:3.5:0.5) and crystallization from ethyl acetate/n-heptane yielded 1.53 g (78%) of the title compound as a colourless solid. m.p. 216-217° C.

### O. Ethyl 3-allyl-2-methyl-6-oxo-1′,3,3′,6,7,9-hexahydrospiro[imidazo[4,5-h]quinoline-8,2′-indene]-5-carboxylate

To a suspension of 3.65 g (8.7 mmol) ethyl 3-allyl-2-methyl-6-oxo-1′,3,3′,4,5,6,7,9-octahydrospiro-[imidazo[4,5-h]quinoline-8,2′-indene]-5-carboxylate in 120 ml ethyl acetate were added 2.1 g (9.3 mmol) 2,3-dichloro-5,6-dicyanobenzoquinone. After 1 h, 250 ml saturated aqueous sodium hydrogen carbonate were added and the mixture was extracted with ethyl acetate. The organic layer was separated, dried over anhydrous sodium sulphate and concentrated in vacuo. Purification of the residue by column chromatography (silica gel, toluene/dioxane/methanol 8:1.5:0.5) and crystallization from ethyl acetate/n-heptane yielded 3.2 g (89%) of the title compound as a pale yellow solid. m.p. 183-183.5° C.

### P. Ethyl 2,3-dimethyl-6-oxo-1′,3,3′,6,7,9-hexahydrospiro[imidazo[4,5-h]quinoline-8,2′-indene]-5-carboxylate

To a solution of 1.5 g (3.8 mmol) ethyl 2,3-dimethyl-6-oxo-1′,3,3′,4,5,6,7,9-octahydrospiro[imidazo[4,5-h]quinoline-8,2′-indene]-5-carboxylate in 50 ml tetrahydrofuran and 5 ml dichloromethane were added 0.86 g (3.8 mmol) 2,3-dichloro-5,6-dicyanobenzoquinone at 0° C. After 4 h, 50 ml 1N aqueous sodium hydroxide were added and the mixture was extracted with dichloromethane. The organic layer was separated, dried over anhydrous sodium sulphate and concentrated in vacuo. Crystallization from diethyl ether yielded 0.95 g (63%) of the title compound. m.p. 205-206° C.

### Q. N,2,3-Trimethyl-6-oxo-1′,3,3′,6,7,9-hexahydrospiro[imidazo[4,5-h]quinoline-8,2′-indene]-5-carboxamide

To a solution of 1.3 g (3.4 mmol) N,2,3-trimethyl-6-oxo-1′,3,3′,4,5,6,7,9-octahydrospiro[imidazo[4,5-h]-quinoline-8,2′-indene]-5-carboxamide in 50 ml tetrahydrofuran were added 0.77 g (3.4 mmol) 2,3-dichloro-5,6-dicyanobenzoquinone at 0° C. After 4 h, 50 ml 1N aqueous sodium hydroxide were added and the mixture was extracted with dichloromethane. The organic layer was separated, dried over anhydrous sodium sulphate and concentrated in vacuo. Crystallization from diethyl ether yielded 0.68 g (53%) of the title compound. m.p. 261-261° C.

### R. Ethyl (2-hydroxy-2,3-dihydro-1H-inden-2-yl)acetate

A solution of lithium diisopropylamide in heptane/tetrahydrofuran (305 ml, 0.55 mol) was cooled to −75° C. and 55 ml (0.56 mol) ethyl acetate were added dropwise while the temperature was kept below −75° C. After addition was complete, the mixture was stirred at −75° C. for 30 min. A solution of 36.5 g (0.27 mol) 2-indanone in 90 ml tetrahydrofuran was added dropwise while the temperature was kept below −75° C. The mixture was stirred for 60 min while the temperature was allowed to rise to −12° C. The mixture was quenched with 200 ml tetrahydrofuran/water (1:1) and 500 ml saturated aqueous ammonium chloride solution were added. The mixture was acidified with 5N hydrochloric acid and extracted with tert-butyl methyl ether (2×500 ml). The organic layer was dried over anhydrous sodium sulphate and concentrated in vacuo. The residue was purified by distillation at 123-130° C. and 0.5 mm Hg, affording 31.2 g (52%) of the title compound. ¹H-NMR (CDCl₃): 1.30 (t, J=7.1 Hz, 3H), 2.76 (s, 2H), 3.02 (d, J=16.1 Hz, 2H), 3.15 (d, J=16.1 Hz, 2H), 3.71 (s, 1H, OH), 4.21 (q, J=7.1 Hz, 2H), 7.18 (m, 4H).

### S. Ethyl [2-(acetylamino)-2,3-dihydro-1H-inden-2-yl]-acetate

A solution of 75 g (0.34 mol) ethyl (2-hydroxy-2,3-dihydro-1H-inden-2-yl)acetate in 1.5 L acetonitrile was cooled to 5° C. 67 ml chlorosulphonic acid were added dropwise while the temperature was allowed to rise to 14° C. The reaction mixture was then stirred for 5 h at ambient temperature. The mixture was poured into water (10 L) and extracted with ethyl acetate. The organic layer was concentrated in vacuo affording 47.7 g (54%) of the title compound. m.p. 72-73° C.

### T. (2-Amino-2,3-dihydro-1H-inden-2-yl)acetic acid hydrochloride

A solution of 23.5 g (90 mmol) ethyl [2-(acetylamino)-2,3-dihydro-1H-inden-2-yl]-acetate in 200 ml 5N hydrochloric acid was refluxed overnight. The mixture was evaporated to dryness and the residue was mixed with ethyl acetate. The obtained solid was collected by filtration affording 12.4 g (61%) of the title compound. m.p. 176-178° C.

### U. Ethyl (2-amino-2,3-dihydro-1H-inden-2-yl)acetate hydrochloride

A solution of 12.4 g (55 mmol) (2-amino-2,3-dihydro-1H-inden-2-yl)acetic acid hydrochloride in 250 ml ethanol was cooled to 0-5° C. and 7.5 ml thionylchloride were added dropwise during 20 min. The mixture was refluxed for 3 h and concentrated in vacuo affording 17 g (quant) of the title compound. m.p. 188-190° C.

### INDUSTRIAL APPLICABILITY

The compounds of the formula 1 and their pharmaceutically acceptable salts (=active compounds according to the invention) have valuable pharmacological properties which make them commercially utilizable. In particular, they exhibit marked inhibition of gastric acid secretion and an excellent gastric and intestinal protective or curative action in warm-blooded animals, in particular humans. In this connection, the active compounds according to the invention are distinguished by a high selectivity of action, a fast onset of action, an advantageous duration of action, efficient control of the duration of action by the dosage, a particularly good antisecretory efficacy, the absence of significant side effects and a large therapeutic range.

“Gastric and intestinal protection or cure” in this connection is understood to include, according to general knowledge, the prevention, the treatment and the maintenance treatment of gastrointestinal diseases, in particular of gastrointestinal inflammatory diseases and lesions (such as, for example, reflux esophagitis, gastritis, hyperacidic or drug-related functional dyspepsia, and peptic ulcer disease [including peptic ulcer bleeding, gastric ulcer, duodenal ulcer]), which can be caused, for example, by microorganisms (e.g. Helicobacter pylori), bacterial toxins, drugs (e.g. certain antiinflammatories and antirheumatics, such as NSAIDs and COX-inhibitors), chemicals (e.g. ethanol), gastric acid or stress situations.

The term “gastrointestinal diseases” is understood to include, according to general knowledge,

A) gastroesophageal reflux disease (GERD), the symptoms of which include, but are not limited to, heartburn and/or acid regurgitation and/or non-add regurgitation.

B) other extra-esophageal manifestations of GERD that include, but are not limited to, acid-related asthma, bronchitis, laryngitis and sleep disorders.

C) other diseases that can be connected to undiagnosed reflux and/or aspiration include, but are not limited to, airway disorders such as asthma, bronchitis, COPD (chronic obstructive pulmonary disease).

D) Helicobacter pylori infection whose eradication is playing a key role in the treatment of gastrointestinal diseases.

E) Furthermore, “gastrointestinal diseases” comprise other gastrointestinal conditions that might be related to acid secretion, such as Zollinger-Ellison syndrome, acute upper gastrointestinal bleeding, nausea, vomiting due to chemotherapy or post-operative conditions, stress ulceration, IBD (inflammatory bowel disease) and particularly IBS (irritable bowel syndrome).

In their excellent properties, the active compounds according to the invention surprisingly prove to be dearly superior to the compounds known from the prior art in various models in which the antiulcerogenic and the antisecretory properties are determined. On account of these properties, the active compounds according to the invention are outstandingly suitable for use in human and veterinary medicine, where they are used, in particular, for the treatment and/or prophylaxis of disorders of the stomach and/or intestine and/or upper digestive tract, particularly of the abovementioned diseases.

A further subject of the invention are therefore the active compounds according to the invention for use in the treatment and/or prophylaxis of the abovementioned diseases.

The invention likewise includes the use of the active compounds according to the invention for the production of medicaments which are employed for the treatment and/or prophylaxis of the above-mentioned diseases.

The invention furthermore includes the use of the active compounds according to the invention for the treatment and/or prophylaxis of the abovementioned diseases.

A further subject of the invention are medicaments which comprise one or more active compounds according to the invention.

As medicaments, the active compounds according to the invention are either employed as such, or preferably in combination with suitable pharmaceutical excipients in the form of tablets, coated tablets (e.g. film-coated tablets), multi unit particulate system tablets, capsules, suppositories, granules, powders (e.g. lyophilized compounds), pellets, patches (e.g. as TTS [transdermal therapeutic system]), emulsions, suspensions or solutions. The content of the active compound is advantageously being between 0.1 and 95 wt % (weight percent in the final dosage form), preferably between 1 and 60 wt %. By means of the appropriate selection of the excipients, it is possible to obtain a pharmaceutical administration form adapted to the active compound and/or to the desired onset and/or duration of action (e.g. a sustained release form or a delayed release form).

The active compounds according to the invention can be administered orally, parenterally (e.g. intravenously), rectally or percutaneously. Oral or intravenous administration is preferred.

The excipients or combinations of excipients which are suitable for the desired pharmaceutical formulations are known to the person skilled in the art on the basis of his/her expert knowledge and are composed of one or more accessory ingredients. In addition to solvents, antioxidants, stabilizers, surfactants, complexing agents (e.g. cyclodextrins), the following excipients may be mentioned as examples: For oral administration, gelling agents, antifoams, plasticizer, adsorbent agents, wetting agents, colorants, flavorings, sweeteners and/or tabletting excipients (e.g. carriers, fillers, binders, disintegrating agents, lubricants, coating agents); for intravenous administration, dispersants, emulsifiers, preservatives, solubilizers, buffer substances and/or isotonic adjusting substances. For percutaneous administration, the person skilled in the art may choose as excipients, for example: solvents, gelling agents, polymers, permeation promoters, adhesives, matrix substances and/or wetting agents.

In general, it has been proven advantageous in human medicine to administer the active compound(s) in the case of oral administration in a daily dose (given continuously or on-demand) of approximately 0.01 to approximately 20, preferably 0.02 to 5, in particular 0.02 to 1.5, mg/kg of body weight, if appropriate in the form of several, preferably 1 to 2, individual doses to achieve the desired result. In the case of a parenteral treatment, similar or (in particular in the case of the intravenous administration of the active compounds), as a rule, lower doses can be used. Furthermore, the frequency of administration can be adapted to intermittent, weekly, monthly, even more infrequent (e.g. implant) dosing. The establishment of the optimal dose and manner of administration of the active compounds necessary in each case can easily be carried out by any person skilled in the art on the basis of his/her expert knowledge.

The medicaments may conveniently be presented in unit dosage form and may be prepared by any of the methods well known in the art of pharmaceutical science. All methods include the step of bringing the active compounds according to the invention into association with the excipients or a combination of excipients. In general the formulations are prepared by uniformly and intimately bringing into association the active compounds according to the invention with liquid excipients or finely divided solid excipients or both and then, if necessary, formulating the product into the desired medicament.

The active compounds according to the invention or their pharmaceutical preparations can also be used in combination with one or more pharmacologically active constituents from other groups of drugs [combination partner(s)]. “Combination” is understood to be the supply of both the active compound(s) according to the invention and the combination partner(s) for separate, sequential, simultaneous or chronologically staggered use. A combination is usually designed with the aim of increasing the principal action in an additive or super-additive sense and/or of eliminating or decreasing the side effects of the combination partner(s), or with the aim to obtain a more rapid onset of action and a fast symptom relief. By choosing the appropriate pharmaceutical formulation of the drugs contained in the combination, the drug release profile of the components can be exactly adapted to the desired effect, e.g. the release of one compound and its onset of action is chronologically previous to the release of the other compound.

A combination can be, for example, a composition containing all active compounds (for example a fixed combination) or a kit-of-parts comprising separate preparations of all active compounds.

A “fixed combination” is defined as a combination wherein a first active ingredient and a second active ingredient are present together in one unit dosage or in a single entity. One example of a “fixed combination” is a pharmaceutical composition wherein the said first active ingredient and the said second active ingredient are present in admixture of simultaneous administration, such as in a formulation. Another example of a “fixed combination” is a pharmaceutical composition wherein the said first active ingredient and the said second active ingredient are present in one unit without being in admixture.

A “kit-of-parts” is defined as a combination wherein the said first active ingredient and the said second active ingredient are present in more than one unit. One example of a “kit-of-parts” is a combination wherein the said first active ingredient and the said second active ingredient are present separately. The components of the kit-of-parts may be administered separately, sequentially, simultaneously or chronologically staggered.

“Other groups of drugs” are understood to include, for example: tranquilizers (for example from the group of the benzodiazepines, like diazepam), spasmolyfics (for example butylscopolaminium bromide [Buscopan®]), anticholinergics (for example atropine sulfate, pirenzepine, tolterodine), pain perception reducing or normalizing agents (for example, paracetamol, tetracaine or procaine or especially oxetacain), and, if appropriate, also enzymes, vitamins, trace elements or amino acids.

To be emphasized in this connection is in particular the combination of the active compounds according to the invention with pharmaceuticals which buffer or neutralize gastric acid (such as, for example, magaldrat, aluminium hydroxide, magnesium carbonate, magnesium hydroxide or other antacids), or especially with pharmaceuticals which inhibit or reduce acid secretion, such as, for example:

(I) histamine-H2 blockers [e.g. cimetidine, ranitidine], or

(II) proton pump inhibitors [e.g. omeprazole, esomeprazole, pantoprazole, lansoprazole, rabeprazole, tenatoprazole, ilaprazole, leminoprazole, all including their salts and enantiomers] or

(III) other potassium-competitive acid blockers [e.g. soraprazan and its stereoisomers, linaprazan, revaprazan, all including their salts]), or

(IV) so-called peripheral anticholinergics (e.g. pirenzepine), with gastrin antagonists such as CCK2 antagonists (cholestocystokinin 2 receptor antagonists).

An important combination to be mentioned is the combination with antibacterially active substances, and especially substances with a bactericidal effect, or combinations thereof. These combination partner(s) are especially useful for the control of Helicobacter pylori infection whose eradication is playing a key role in the treatment of gastrointestinal diseases. As suitable antibacterially active combination partner(s) may be mentioned, for example:

(A) cephalosporins, such as, for example, cifuroximaxetil

(B) penicillins, such as, for example, amoxicillin, ampicillin

(C) tetracyclines, such as, for example, tetracycline itself, doxycydine

(D) β-lactamase inhibitors, such as, for example, clavulanic acid

(E) macrolide antibiotics, such as, for example, erythromycin, clarithromycin, azithromycin

(F) rifamycines, such as, for example, rifamycine itself

(G) glycoside antibiotics, such as, for example, gentamicin, streptomycin

(H) gyrase inhibitors, such as, for example, ciprofloxacin, gatifloxacin, moxifloxacin

(I) oxazolidines, such as, for example, linezolid

(J) nitrofurans or nitroimidazoles, such as, for example, metronidazole, tinidazole, nitrofurantoin

(K) bismuth salts, such as, for example, bismuth subcitrat

(L) other antibacterially active substances

and combinations of substances selected from (A) to (L), for example darithromycin+metronidazole. Preferred is the use of two combination partners. Preferred is the use of two combination partners selected from amoxicillin, clarithromycin and metronidazole. A preferred example is the use of amoxicillin and clarithromycin.

In view of their excellent activity regarding gastric and intestinal protection or cure, the active compounds according to the invention are especially suited for a free or fixed combination with drugs, which are known to cause “drug-induced dyspepsia” or are known to have a certain ulcerogenic potency, such as, for example, acetylsalicylic acid, certain antiinflammatories and antirheumatics, such as NSAIDs (non-steroidal antiinflammatory drugs, e.g. etofenamate, diclofenac, indometacin, ibuprofen, piroxicam, naproxen, meloxicam), oral steroids, bisphosphonates (e.g. alendronate), or even NO-releasing NSAIDs, COX-2 inhibitors (e.g. celecoxib, lumiracoxib).

In addition, the active compounds according to the invention are suited for a free or fixed combination with motility-modifying or -regulating drugs (e.g. gastroprokinetics like mosapride, tegaserod, itopride, metoclopramid), and especially with pharmaceuticals which reduce or normalize the incidence of transient lower esophageal sphincter relaxation (TLESR), such as, for example, GABA-B agonists (e.g. baclofen, (2R)-3-amino-2-fluoropropylphosphinic acid) or allosteric GABA-B agonists (e.g. 3,5-bis(1,1-dimethylethyl-4-hydroxy-β,β-dimethylbenzenepropanol), GABA-B re-uptake inhibitors (e.g. tiagabine), metabotropic glutamate receptor type 5 (mGluR5) antagonists (e.g. 2-methyl-6-(phenylethynyl)pyridine hydrochloride), CB2 (cannabinoid receptor) agonists (e.g. [(3R)2,3-dihydro-5-methyl-3-(4-morpholinyl-methyl)pyrrolo[1,2,3,de]-1,4-benzoxazin-6-y]-1-naphthalenylmethanone mesylate). Pharmaceuticals used for the treatment of IBS or IBD are also suitable combination partner(s), such as, for example: 5-HT4 receptor agonists like mosapride, tegaserod; 5-HT3 receptor antagonists like alosetron, cilansetron; NK2 antagonists like saredutant, nepadutant; κ-opiate agonists like fedotozine.

Suitable combination partner(s) also comprise airway therapeutics, for example for the treatment of acid-related asthma and bronchitis. In some cases, the use of a hypnotic aid (such as, for example, Zolpidem [Bikalm®]) as combination partner(s) may be rational, for example for the treatment of GERD-induced sleep disorders.

1. A compound of the formula 1 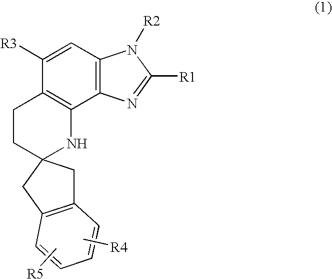
in which
R1 is hydrogen, 1-4C-alkyl, 3-7C-cycloalkyl, 3-7C-cycloalkyl-1-4C-alkyl, 1-4C-alkoxy, 1-4C-alkoxy-1-4C-alkyl, 1-4C-alkoxycarbonyl, 2-4C-alkenyl, 2-4C-alkynyl, fluoro-1-4C-alkyl or hydroxy-1-4C-alkyl,
R2 is hydrogen, 1-4C-alkyl, 1-4C-alkoxy, 3-7C-cycloalkyl, 3-7C-cycloalkyl-1-4C-alkyl, hydroxy-1-4C-alkyl, 1-4C-alkoxy-1-4C-alkyl, 2-4C-alkenyl, 2-4C-alkynyl or fluoro-1-4C-alkyl,
R3 is hydrogen, halogen, fluoro-1-4C-alkyl, 1-4C-alkyl, 2-4C-alkenyl, 2-4C-alkynyl, carboxyl, 1-4C-alkoxycarbonyl, hydroxy-1-4C-alkyl, 1-4C-alkoxy-1-4C-alkyl, 1-4C-alkoxy-1-4C-alkoxy-1-4C-alkyl, fluoro-1-4C-alkoxy-1-4C-alkyl or the group —CO—NR31R32,
where
R31 is hydrogen, hydroxyl, 1-7C-alkyl, 3-7C-cycloalkyl, hydroxy-1-4C-alkyl, 1-4C-alkoxy-1-4C-alkyl, 1-4C-alkylcarbonyl-1-4C-alkyl, 1-4C-alkylcarbonyl or 1-4C-alkoxycarbonyl and
R32 is hydrogen, 1-7C-alkyl, hydroxy-1-4C-alkyl, or 1-4C-alkoxy-1-4C-alkyl,
or where
R31 and R32 together, including the nitrogen atom to which both are bonded, are a pyrrolidino, hydroxypyrrolidino, piperidino, piperazino, azetidino, hydroxyazetidino, fluorazetidino, aziridino, N-1-4C-alkylpiperazino or morpholino group,
R4 and R5 are identical or different substituents selected from the group consisting of hydrogen, 1-4C-alkyl, hydroxy-1-4C-alkyl, 1-4C-alkoxy, 1-4C-alkoxy-1-4C-alkyl, hydroxy-1-4C-alkoxy, 1-4C-alkoxy-1-4C-alkoxy, 2-4C-alkenyloxy, 1-4C-alkylcarbonyl, carboxyl, 1-4C-alkoxycarbonyl, carboxy-1-4C-alkyl, 1-4C-alkoxycarbonyl-1-4C-alkyl, halogen, hydroxyl, trifluoromethyl, halo-1-4C-alkoxy, nitro, amino, mono- or di-1-4C-alkylamino, 1-4C-alkylcarbonylamino, 1-4C-alkoxycarbonylamino, 1-4C-alkoxy-1-4C-alkoxycarbonylamino and sulfonyl,
or a salt thereof.

2. A compound of the formula 1 as claimed in claim 1, in which
R1 is hydrogen, 1-4C-alkyl or hydroxy-1-4C-alkyl,
R2 is hydrogen, 1-4C-alkyl, hydroxy-1-4C-alkyl or 2-4C-alkenyl,
R3 is carboxyl, 1-4C-alkoxycarbonyl, hydroxy-1-4C-alkyl, 1-4C-alkoxy-1-4C-alkyl or the group —CO—NR31R32,
where
R31 is hydrogen, hydroxyl, 1-7C-alkyl, 3-7C-cycloalkyl, hydroxy-1-4C-alkyl, 1-4C-alkoxy-1-4C-alkyl or 1-4C-alkylcarbonyl-1-4C-alkyl and
R32 is hydrogen, 1-7C-alkyl, hydroxy-1-4C-alkyl or 1-4C-alkoxy-1-4C-alkyl,
or where
R31 and R32 together, including the nitrogen atom to which both are bonded, are a pyrrolidino, hydroxypyrrolidino, piperidino, piperazino, azetidino, hydroxyazetidino, fluorazetidino, aziridino, N-1-4C-alkylpiperazino or morpholino group,
R4 and R5 are identical or different substituents selected from the group consisting of hydrogen, 1-4C-alkyl and halogen,
or a salt thereof.

3. A compound of the formula 1 as claimed in claim 1, in which
R1 is 1-4C-alkyl,
R2 is hydrogen, 1-4C-alkyl or 2-4C-alkenyl,
R3 is carboxyl, 1-4C-alkoxycarbonyl, hydroxy-1-4C-alkyl or the group —CO—NR31R32,
where
R31 is hydrogen, 1-7C-alkyl, 3-7C-cycloalkyl, hydroxy-1-4C-alkyl, 1-4C-alkoxy-1-4C-alkyl or 1-4C-alkylcarbonyl-1-4C-alkyl and
R32 is hydrogen or 1-7C-alkyl,
or where
R31 and R32 together, including the nitrogen atom to which both are bonded, are a pyrrolidino, azetidino, hydroxyazetidino, fluorazetidino or morpholino group,
R4 and R5 are each hydrogen,
or a salt thereof.

4. A compound of the formula 1 as claimed in claim 1, in which
R1 is 1-4C-alkyl,
R2 is 1-4C-alkyl,
R3 is the group —CO—NR31R32,
where
R31 is hydrogen, 1-7C-alkyl, 3-7C-cycloalkyl, 1-4C-alkoxy-1-4C-alkyl or 1-4C-alkylcarbonyl-1-4C-alkyl and
R32 is hydrogen or 1-7C-alkyl,
or where
R31 and R32 together, including the nitrogen atom to which both are bonded, are a pyrrolidino, azetidino, fluorazetidino or morpholino group,
R4 and R5 are each hydrogen,
or a salt thereof.

5. A compound of the formula 1 as claimed in claim 1, in which
R1 is 1-4C-alkyl,
R2 is 1-4C-alkyl,
R3 is carboxyl, 1-4C-alkoxycarbonyl or the group —CO—NR31R32,
where
R31 is hydrogen, hydroxy-1-4C-alkyl or 1-7C-alkyl,
R32 is hydrogen or 1-7C-alkyl and
R4 and R5 are each hydrogen,
or a salt thereof.

6. A compound of the formula 1 as claimed in claim 1, in which
R1 is methyl,
R2 is methyl,
R3 is the group —CO—NR31R32,
where
R31 is hydrogen, methyl, cyclopropyl, 2-methoxyethyl, 3-methoxypropyl or 2-oxo-propyl and
R32 is hydrogen or methyl,
or where
R31 and R32 together, including the nitrogen atom to which both are bonded, are a pyrrolidino, azetidino, 3-fluorazetidino or morpholino group,
R4 and R5 are each hydrogen,
or a salt thereof.

7. A compound of the formula 1 as claimed in claim 1, in which the substituents R1, R2, R3, R4 and R5 have the meanings given in the following table, whereby Me is CH₃ and Et is C₂H₅

| R1 | R2 | R3 | R4 | R5 |
|---|---|---|---|---|
| Me | Me | CH₂OH | H | H |
| Me | Me | CH₂OCH₃ | H | H |
| Me | Me | C(O)NHMe | H | H |
| Me | Me | C(O)N-pyrrolidine | H | H |
| Me | Me | C(O)NH(CH₂)₂OH | H | H |
| Me | Me | C(O)NH(CH₂)₂OMe | H | H |
| Me | Me | C(O)NH₂ | H | H |
| Me | Me | C(O)N-morpholine | H | H |
| Me | Me | C(O)NMe₂ | H | H |
| Me | Me | C(O)N-aziridine | H | H |
| Me | Me | C(O)OEt | H | H |
| Me | Me | C(O)OH | H | H |
| Me | Me | C(O)N-azetidine | H | H |
| Me | Me | C(O)NH(CH₂)₂Me | H | H |
| Me | Me | C(O)NHCH₂CH(OH)CH₂OH | H | H |
| Me | Me | C(O)NH-cyclopropyl | H | H |
| Me | Me | H | H | H |
| Me | Me | C(O)NHEt | H | H |
| Me | Me | C(O)NH(CH₂)₃OH | H | H |
| Me | Me | C(O)NH(CH₂)₃OMe | H | H |
| Me | Me | C(O)NHCH₂C(O)CH₃ | H | H |
| Me | Me | C(O)N-3-fluorazetidine | H | H |
| Me | Me | C(O)N(CH₃)—(CH₂)₂OH | H | H |
| Me | Me | C(O)N(CH₃)—(CH₂)₂OMe | H | H |
| Me | Me | C(O)N(CH₃)—(CH₂)₃OH | H | H |
| Me | Me | C(O)N(CH₃)—(CH₂)₃OMe | H | H |
| Me | H | CH₂OH | H | H |
| Me | H | CH₂OCH₃ | H | H |
| Me | H | C(O)NHMe | H | H |
| Me | H | C(O)N-pyrrolidine | H | H |
| Me | H | C(O)NH(CH₂)₂OH | H | H |
| Me | H | C(O)NH(CH₂)₂OMe | H | H |
| Me | H | C(O)NH₂ | H | H |
| Me | H | C(O)N-morpholine | H | H |
| Me | H | C(O)NMe₂ | H | H |
| Me | H | C(O)N-aziridine | H | H |
| Me | H | C(O)OEt | H | H |
| Me | H | C(O)OH | H | H |
| Me | H | C(O)N-azetidine | H | H |
| Me | H | C(O)NH(CH₂)₂Me | H | H |
| Me | H | C(O)NHCH₂CH(OH)CH₂OH | H | H |
| Me | H | C(O)NH-cyclopropyl | H | H |
| Me | H | H | H | H |
| Me | H | C(O)NHEt | H | H |
| Me | H | C(O)NH(CH₂)₃OH | H | H |
| Me | H | C(O)NH(CH₂)₃OMe | H | H |
| Me | H | C(O)NHCH₂C(O)CH₃ | H | H |
| Me | H | C(O)N-3-fluorazetidine | H | H |
| Me | H | C(O)N(CH₃)—(CH₂)₂OH | H | H |
| Me | H | C(O)N(CH₃)—(CH₂)₂OMe | H | H |
| Me | H | C(O)N(CH₃)—(CH₂)₃OH | H | H |
| Me | H | C(O)N(CH₃)—(CH₂)₃OMe | H | H |

or a salt thereof.

8. A compound of the formula 1 as claimed in claim 1, which is selected from the group consisting of
Ethyl 2,3-dimethyl-1′,3,3′,6,7,9-hexahydrospiro[imidazo[4,5-h]quinoline-8,2′-indene]-5-carboxylate,
Methyl 3-allyl-2-methyl-1′,3,3′,6,7,9-hexahydrospiro[imidazo[4,5-h]quinoline-8,2′-indene]-5-carboxylate,
Ethyl 3-allyl-2-methyl-1′,3,3′,6,7,9-hexahydrospiro[imidazo[4,5-h]quinoline-8,2′-indene]-5-carboxylate,
N,2,3-Trimethyl-1′,3,3′,6,7,9-hexahydrospiro[imidazo[4,5-h]quinoline-8,2′-indene]-5-carboxamide,
2,3-Dimethyl-1′,3,3′,6,7,9-hexahydrospiro[imidazo[4,5-h]quinoline-8,2′-indene]-5-carboxylic acid,
(2,3-Dimethyl-1′,3,3′,6,7,9-hexahydrospiro[imidazo[4,5-h]quinoline-8,2′-inden]-5-yl)methanol,
3-Allyl-N,2-dimethyl-1′,3,3′,6,7,9-hexahydrospiro[imidazo[4,5-h]quinoline-8,2′-indene]-5-carboxamide,
3-Allyl-N,N,2-dimethyl-1′,3,3′,6,7,9-hexahydrospiro[imidazo[4,5-h]quinoline-8,2′-indene]-5-carboxamide,
N,2-dimethyl-1′,3,3′,6,7,9-hexahydrospiro[imidazo[4,5-h]quinoline-8,2′-indene]-5-carboxamide,
N,N,2-Trimethyl-1′,3,3′,6,7,9-hexahydrospiro[imidazo[4,5-h]quinoline-8,2′-indene]-5-carboxamide,
N,N,2,3-tetramethyl-1′,3,3′,6,7,9-hexahydrospiro[imidazo[4,5-h]quinoline-8,2′-indene]-5-carboxamide,
N-(2-hydroxyethyl)-N,2,3-trimethyl-1′,3,3′,6,7,9-hexahydrospiro[imidazo[4,5-h]quinoline-8,2′-indene]-5-carboxamide,
1-[(2,3-dimethyl-1′,3,3′,6,7,9-hexahydrospiro[imidazo[4,5-h]quinoline-8,2′-inden]-5-yl)carbonyl]azetidin-3-ol,
2,3-dimethyl-N-(2-oxopropyl)-1′,3,3′,6,7,9-hexahydrospiro[imidazo[4,5-h]quinoline-8,2′-indene]-5-carboxamide,
5-[(3-fluoroazetidin-1-yl)carbonyl]-2,3-dimethyl-1′,3,3′,6,7,9-hexahydrospiro[imidazo[4,5-h]quinoline-8,2′-indene],
N-(2-methoxyethyl)-2,3-dimethyl-1′,3,3′,6,7,9-hexahydrospiro[imidazo[4,5-h]quinoline-8,2′-indene]-5-carboxamide,
N-(2-hydroxyethyl)-2,3-dimethyl-1′,3,3′,6,7,9-hexahydrospiro[imidazo[4,5-h]quinoline-8,2′-indene]-5-carboxamide,
2,3-dimethyl-1′,3,3′,6,7,9-hexahydrospiro[imidazo[4,5-h]quinoline-8,2′-indene]-5-carboxamide,
N-[(2S)-2-hydroxypropyl]-N,2,3-trimethyl-1′,3,3′,6,7,9-hexahydrospiro[imidazo[4,5-h]quinoline-8,2′-indene]-5-carboxamide,
N-[(2S)-2-hydroxypropyl]-2,3-dimethyl-1′,3,3′,6,7,9-hexahydrospiro[imidazo[4,5-h]quinoline-8,2′-indene]-5-carboxamide,
N-(2-methoxyethyl)-N,2,3-trimethyl-1′,3,3′,6,7,9-hexahydrospiro[imidazo[4,5-h]quinoline-8,2′-indene]-5-carboxamide,
2,3-dimethyl-5-(pyrrolidin-1-ylcarbonyl)-1′,3,3′,6,7,9-hexahydrospiro[imidazo[4,5-h]quinoline-8,2′-indene],
5-(azetidin-1-ylcarbonyl)-2,3-dimethyl-1′,3,3′,6,7,9-hexahydrospiro[imidazo[4,5-h]quinoline-8,2′-indene],
N-cyclopropyl-2,3-dimethyl-1′,3,3′,6,7,9-hexahydrospiro[imidazo[4,5-h]quinoline-8,2′-indene]-5-carboxamide,
2,3-dimethyl-5-(morpholin-4-ylcarbonyl)-1′,3,3′,6,7,9-hexahydrospiro[imidazo[4,5-h]quinoline-8,2′-indene],
N-(3-hydroxypropyl)-2,3-dimethyl-1′,3,3′,6,7,9-hexahydrospiro[imidazo[4,5-h]quinoline-8,2′-indene]-5-carboxamide,
N-(3-methoxypropyl)-2,3-dimethyl-1′,3,3′,6,7,9-hexahydrospiro[imidazo[4,5-h]quinoline-8,2′-indene]-5-carboxamide,
N,2,3-Trimethyl-1′,3,3′,6,7,9-hexahydrospiro[imidazo[4,5-h]quinoline-8,2′-indene]-5-carboxamide methanesulphonate,
N,2,3-Trimethyl-1′,3,3′,6,7,9-hexahydrospiro[imidazo[4,5-h]quinoline-8,2′-indene]-5-carboxamide hydrochloride,
N,2,3-Trimethyl-1′,3,3′,6,7,9-hexahydrospiro[imidazo[4,5-h]quinoline-8,2′-indene]-5-carboxamide malonate,
N-(2-ethoxyethyl)-2,3-dimethyl-1′,3,3′,6,7,9-hexahydrospiro[imidazo[4,5-h]quinoline-8,2′-indene]-5-carboxamide
N-ethyl-2,3-dimethyl-1′,3,3′,6,7,9-hexahydrospiro[imidazo[4,5-h]quinoline-8,2′-indene]-5-carboxamide,
and salts thereof.

9. A compound of the formula 1 as claimed in claim 1, which is selected from the group consisting of
N,2,3-Trimethyl-1′,3,3′,6,7,9-hexahydrospiro[imidazo[4,5-h]quinoline-8,2′-indene]-5-carboxamide,
N,N,2,3-tetramethyl-1′,3,3′,6,7,9-hexahydrospiro[imidazo[4,5-h]quinoline-8,2′-indene]-5-carboxamide,
2,3-dimethyl-N-(2-oxopropyl)-1′,3,3′,6,7,9-hexahydrospiro[imidazo[4,5-h]quinoline-8,2′-indene]-5-carboxamide,
5-[(3-fluoroazetidin-1-yl)carbonyl]-2,3-dimethyl-1′,3,3′,6,7,9-hexahydrospiro[imidazo[4,5-h]quinoline-8,2′-indene],
N-(2-methoxyethyl)-2,3-dimethyl-1′,3,3′,6,7,9-hexahydrospiro[imidazo[4,5-h]quinoline-8,2′-indene]-5-carboxamide,
2,3-dimethyl-1′,3,3′,6,7,9-hexahydrospiro[imidazo[4,5-h]quinoline-8,2′-indene]-5-carboxamide,
N-(2-methoxyethyl)-N,2,3-trimethyl-1′,3,3′,6,7,9-hexahydrospiro[imidazo[4,5-h]quinoline-8,2′-indene]-5-carboxamide,
2,3-dimethyl-5-(pyrrolidin-1-ylcarbonyl)-1′,3,3′,6,7,9-hexahydrospiro[imidazo[4,5-h]quinoline-8,2′-indene],
5-(azetidin-1-ylcarbonyl)-2,3-dimethyl-1′,3,3′,6,7,9-hexahydrospiro[imidazo[4,5-h]quinoline-8,2′-indene],
N-cyclopropyl-2,3-dimethyl-1′,3,3′,6,7,9-hexahydrospiro[imidazo[4,5-h]quinoline-8,2′-indene]-5-carboxamide,
2,3-dimethyl-5-(morpholin-4-ylcarbonyl)-1′,3,3′,6,7,9-hexahydrospiro[imidazo[4,5-h]quinoline-8,2′-indene]
N-(3-methoxypropyl)-2,3-dimethyl-1′,3,3′,6,7,9-hexahydrospiro[imidazo[4,5-h]quinoline-8,2′-indene]-5-carboxamide,
and salts thereof.

10. A compound of the formula 4, 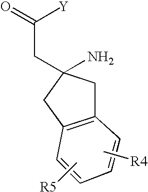
in which R4 and R5 are defined as in claim 1 and Y is a suitable leaving group.

11. A compound of the formula 5, 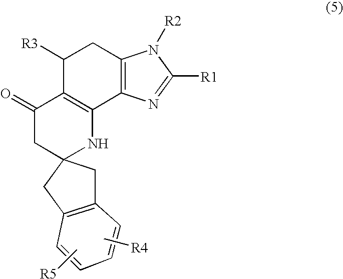
in which
R1, R2, R3, R4 and R5 are defined as in claim 1.

12. A compound of the formula 2, 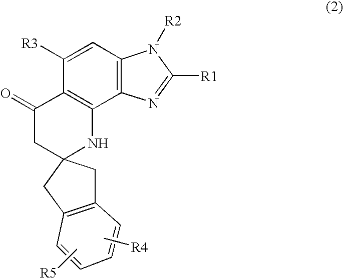
R1, R2, R3, R4 and R5 are defined as in claim 1.

13. (canceled)

14. A pharmaceutical composition comprising one or more compounds according to claim 1 and/or a pharmaceutically acceptable salt thereof together with a pharmaceutically acceptable auxiliary and/or excipient.

15. (canceled)

16. A pharmaceutical composition comprising one or more compounds according to claim 10 and/or a pharmaceutically acceptable salt thereof together with a pharmaceutically acceptable auxiliary and/or excipient.

17. A pharmaceutical composition comprising one or more compounds according to claim 11 and/or a pharmaceutically acceptable salt thereof together with a pharmaceutically acceptable auxiliary and/or excipient.

18. A pharmaceutical composition comprising one or more compounds according to claim 12 and/or a pharmaceutically acceptable salt thereof together with a pharmaceutically acceptable auxiliary and/or excipient.

19. A method for preventing and/or treating gastrointestinal disorders in a patient comprising administering to a patient in need thereof a compound according to claim 1 or a pharmaceutically acceptable salt thereof.

20. A method for preventing and/or treating gastrointestinal disorders in a patient comprising administering to a patient in need thereof a compound according to claim 10 or a pharmaceutically acceptable salt thereof.

21. A method for preventing and/or treating gastrointestinal disorders in a patient comprising administering to a patient in need thereof a compound according to claim 11 or a pharmaceutically acceptable salt thereof.

22. A method for preventing and/or treating gastrointestinal disorders in a patient comprising administering to a patient in need thereof a compound according to claim 12 or a pharmaceutically acceptable salt thereof.
